SEQUENCE LISTING (373 sequences)

SEQ ID NO: 1 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2-8 - skipped (7 entries)

SEQ ID NO: 9 - protein, 385 aa, Artificial Sequence
  BSG
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 10-13 - skipped (4 entries)

SEQ ID NO: 14 - protein, 613 aa, Artificial Sequence
  IGSF8
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 15-20 - skipped (6 entries)

SEQ ID NO: 21 - protein, 748 aa, Artificial Sequence
  ITGB1
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 22 - protein, 1032 aa, Artificial Sequence [sequence omitted]

SEQ ID NO: 23 - protein, 630 aa, Artificial Sequence
  SLC3A2
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 24-32 - skipped (9 entries)

SEQ ID NO: 33 - protein, 192 aa, Artificial Sequence
  PTGFRN Fragment
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

SEQ ID NO: 34-204 - skipped (171 entries)

SEQ ID NO: 205 - protein, 4 aa, Artificial Sequence
  Synthetic construct
KKKK

SEQ ID NO: 206 - protein, 5 aa, Artificial Sequence
  Synthetic construct
KKKKK

SEQ ID NO: 207 - protein, 4 aa, Artificial Sequence
  Synthetic construct
RRRR

SEQ ID NO: 208 - protein, 5 aa, Artificial Sequence
  Synthetic construct
RRRRR

SEQ ID NO: 209 - protein, 4 aa, Artificial Sequence
  Synthetic construct
  MOD_RES (1)..(4): X is Lysine or Arginine
XXXX

SEQ ID NO: 210 - protein, 5 aa, Artificial Sequence
  Synthetic construct
  MOD_RES (1)..(5): X is Lysine or Arginine
XXXXX

SEQ ID NO: 211 - protein, 7 aa, Artificial Sequence
  Synthetic construct
GGKLSKK

SEQ ID NO: 212 - protein, 7 aa, Artificial Sequence
  Synthetic construct
GAKLSKK

SEQ ID NO: 213 - protein, 7 aa, Artificial Sequence
  Synthetic construct
GGKQSKK

SEQ ID NO: 214 - protein, 7 aa, Artificial Sequence
  Synthetic construct
GGKLAKK

SEQ ID NO: 215 - protein, 6 aa, Artificial Sequence
  Synthetic construct
GGKLSK

SEQ ID NO: 216 - protein, 6 aa, Artificial Sequence
  Synthetic construct
GAKLSK

SEQ ID NO: 217 - protein, 6 aa, Artificial Sequence
  Synthetic construct
GGKQSK

SEQ ID NO: 218 - protein, 6 aa, Artificial Sequence
  Synthetic construct
GGKLAK

SEQ ID NO: 219 - protein, 4 aa, Artificial Sequence
  Synthetic construct
KKKG

SEQ ID NO: 220 - protein, 5 aa, Artificial Sequence
  Synthetic construct
KKKGY

SEQ ID NO: 221 - protein, 6 aa, Artificial Sequence
  Synthetic construct
KKKGYN

SEQ ID NO: 222 - protein, 7 aa, Artificial Sequence
  Synthetic construct
KKKGYNV

SEQ ID NO: 223 - protein, 8 aa, Artificial Sequence
  Synthetic construct
KKKGYNVN

SEQ ID NO: 224 - protein, 6 aa, Artificial Sequence
  Synthetic construct
KKKGYS

SEQ ID NO: 225 - protein, 6 aa, Artificial Sequence
  Synthetic construct
KKKGYG

SEQ ID NO: 226 - protein, 7 aa, Artificial Sequence
  Synthetic construct
KKKGYGG

SEQ ID NO: 227 - protein, 5 aa, Artificial Sequence
  Synthetic construct
KKKGS

SEQ ID NO: 228 - protein, 6 aa, Artificial Sequence
  Synthetic construct
KKKGSG

SEQ ID NO: 229 - protein, 7 aa, Artificial Sequence
  Synthetic construct
KKKGSGS

SEQ ID NO: 230 - protein, 4 aa, Artificial Sequence
  Synthetic construct
KKKS

SEQ ID NO: 231 - protein, 5 aa, Artificial Sequence
  Synthetic construct
KKKSG

SEQ ID NO: 232 - protein, 6 aa, Artificial Sequence
  Synthetic construct
KKKSGG

SEQ ID NO: 233 - protein, 7 aa, Artificial Sequence
  Synthetic construct
KKKSGGS

SEQ ID NO: 234 - protein, 8 aa, Artificial Sequence
  Synthetic construct
KKKSGGSG

SEQ ID NO: 235 - protein, 8 aa, Artificial Sequence
  Synthetic construct
KKSGGSGG

SEQ ID NO: 236 - protein, 10 aa, Artificial Sequence
  Synthetic construct
KKKSGGSGGS

SEQ ID NO: 237 - protein, 8 aa, Artificial Sequence
  Synthetic construct
KRFSFKKS

SEQ ID NO: 238 - protein, 8 aa, Artificial Sequence
  Synthetic construct
GGKLSKKK

SEQ ID NO: 239 - protein, 8 aa, Artificial Sequence
  Synthetic construct
GGKLSKKS

SEQ ID NO: 240 - protein, 8 aa, Artificial Sequence
  Synthetic construct
GAKLSKKK

SEQ ID NO: 241 - protein, 8 aa, Artificial Sequence
  Synthetic construct
GAKLSKKS

SEQ ID NO: 242 - protein, 8 aa, Artificial Sequence
  Synthetic construct
GGKQSKKK

SEQ ID NO: 243 - protein, 8 aa, Artificial Sequence
  Synthetic construct
GGKQSKKS

SEQ ID NO: 244 - protein, 8 aa, Artificial Sequence
  Synthetic construct
GGKLAKKK

SEQ ID NO: 245 - protein, 8 aa, Artificial Sequence
  Synthetic construct
GGKLAKKS

SEQ ID NO: 246 - protein, 14 aa, Artificial Sequence
  Synthetic construct
GGKLSKKKKGYNVN

SEQ ID NO: 247 - protein, 14 aa, Artificial Sequence
  Synthetic construct
GAKLSKKKKGYNVN

SEQ ID NO: 248 - protein, 14 aa, Artificial Sequence
  Synthetic construct
GGKQSKKKKGYNVN

SEQ ID NO: 249 - protein, 14 aa, Artificial Sequence
  Synthetic construct
GGKLAKKKKGYNVN

SEQ ID NO: 250 - protein, 14 aa, Artificial Sequence
  Synthetic construct
GGKLSKKKKGYSGG

SEQ ID NO: 251 - protein, 14 aa, Artificial Sequence
  Synthetic construct
GGKLSKKKKGSGGS

SEQ ID NO: 252 - protein, 14 aa, Artificial Sequence
  Synthetic construct
GGKLSKKKKSGGSG

SEQ ID NO: 253 - protein, 14 aa, Artificial Sequence
  Synthetic construct
GGKLSKKKSGGSGG

SEQ ID NO: 254 - protein, 14 aa, Artificial Sequence
  Synthetic construct
GGKLSKKSGGSGGS

SEQ ID NO: 255 - protein, 14 aa, Artificial Sequence
  Synthetic construct
GGKLSKSGGSGGSV

SEQ ID NO: 256 - skipped

SEQ ID NO: 257 - protein, 29 aa, Artificial Sequence
  Synthetic construct
GGKLSKKKKGYNVNDEKAKEKDKKAEGAA

SEQ ID NO: 258 - protein, 28 aa, Artificial Sequence
  Synthetic construct
GGKLSKKKKGYNVNDEKAKEKDKKAEGA

SEQ ID NO: 259 - protein, 27 aa, Artificial Sequence
  Synthetic construct
GGKLSKKKKGYNVNDEKAKEKDKKAEG

SEQ ID NO: 260 - protein, 26 aa, Artificial Sequence
  Synthetic construct
GGKLSKKKKGYNVNDEKAKEKDKKAE

SEQ ID NO: 261 - protein, 25 aa, Artificial Sequence
  Synthetic construct
GGKLSKKKKGYNVNDEKAKEKDKKA

SEQ ID NO: 262 - protein, 24 aa, Artificial Sequence
  Synthetic construct
GGKLSKKKKGYNVNDEKAKEKDKK

SEQ ID NO: 263 - protein, 23 aa, Artificial Sequence
  Synthetic construct
GGKLSKKKKGYNVNDEKAKEKDK

SEQ ID NO: 264 - protein, 22 aa, Artificial Sequence
  Synthetic construct
GGKLSKKKKGYNVNDEKAKEKD

SEQ ID NO: 265 - protein, 21 aa, Artificial Sequence
  Synthetic construct
GGKLSKKKKGYNVNDEKAKEK

SEQ ID NO: 266 - protein, 20 aa, Artificial Sequence
  Synthetic construct
GGKLSKKKKGYNVNDEKAKE

SEQ ID NO: 267 - protein, 19 aa, Artificial Sequence
  Synthetic construct
GGKLSKKKKGYNVNDEKAK

SEQ ID NO: 268 - protein, 18 aa, Artificial Sequence
  Synthetic construct
GGKLSKKKKGYNVNDEKA

SEQ ID NO: 269 - protein, 17 aa, Artificial Sequence
  Synthetic construct
GGKLSKKKKGYNVNDEK

SEQ ID NO: 270 - protein, 16 aa, Artificial Sequence
  Synthetic construct
GGKLSKKKKGYNVNDE

SEQ ID NO: 271 - protein, 15 aa, Artificial Sequence
  Synthetic construct
GGKLSKKKKGYNVND

SEQ ID NO: 272 - protein, 13 aa, Artificial Sequence
  Synthetic construct
GGKLSKKKKGYNV

SEQ ID NO: 273 - protein, 12 aa, Artificial Sequence
  Synthetic construct
GGKLSKKKKGYN

SEQ ID NO: 274 - protein, 11 aa, Artificial Sequence
  Synthetic construct
GGKLSKKKKGY

SEQ ID NO: 275 - protein, 10 aa, Artificial Sequence
  Synthetic construct
GGKLSKKKKG

SEQ ID NO: 276 - protein, 9 aa, Artificial Sequence
  Synthetic construct
GGKLSKKKK

SEQ ID NO: 277 - protein, 28 aa, Artificial Sequence
  Synthetic construct
GAKKSKKRFSFKKSFKLSGFSFKKNKKE

SEQ ID NO: 278 - protein, 27 aa, Artificial Sequence
  Synthetic construct
GAKKSKKRFSFKKSFKLSGFSFKKNKK

SEQ ID NO: 279 - protein, 26 aa, Artificial Sequence
  Synthetic construct
GAKKSKKRFSFKKSFKLSGFSFKKNK

SEQ ID NO: 280 - protein, 25 aa, Artificial Sequence
  Synthetic construct
GAKKSKKRFSFKKSFKLSGFSFKKN

SEQ ID NO: 281 - protein, 24 aa, Artificial Sequence
  Synthetic construct
GAKKSKKRFSFKKSFKLSGFSFKK

SEQ ID NO: 282 - protein, 23 aa, Artificial Sequence
  Synthetic construct
GAKKSKKRFSFKKSFKLSGFSFK

SEQ ID NO: 283 - protein, 22 aa, Artificial Sequence
  Synthetic construct
GAKKSKKRFSFKKSFKLSGFSF

SEQ ID NO: 284 - protein, 21 aa, Artificial Sequence
  Synthetic construct
GAKKSKKRFSFKKSFKLSGFS

SEQ ID NO: 285 - protein, 20 aa, Artificial Sequence
  Synthetic construct
GAKKSKKRFSFKKSFKLSGF

SEQ ID NO: 286 - protein, 19 aa, Artificial Sequence
  Synthetic construct
GAKKSKKRFSFKKSFKLSG

SEQ ID NO: 287 - protein, 18 aa, Artificial Sequence
  Synthetic construct
GAKKSKKRFSFKKSFKLS

SEQ ID NO: 288 - protein, 17 aa, Artificial Sequence
  Synthetic construct
GAKKSKKRFSFKKSFKL

SEQ ID NO: 289 - protein, 16 aa, Artificial Sequence
  Synthetic construct
GAKKSKKRFSFKKSFK

SEQ ID NO: 290 - protein, 15 aa, Artificial Sequence
  Synthetic construct
GAKKSKKRFSFKKSF

SEQ ID NO: 291 - protein, 14 aa, Artificial Sequence
  Synthetic construct
GAKKSKKRFSFKKS

SEQ ID NO: 292 - protein, 13 aa, Artificial Sequence
  Synthetic construct
GAKKSKKRFSFKK

SEQ ID NO: 293 - protein, 12 aa, Artificial Sequence
  Synthetic construct
GAKKSKKRFSFK

SEQ ID NO: 294 - protein, 11 aa, Artificial Sequence
  Synthetic construct
GAKKSKKRFSF

SEQ ID NO: 295 - protein, 10 aa, Artificial Sequence
  Synthetic construct
GAKKSKKRFS

SEQ ID NO: 296 - protein, 9 aa, Artificial Sequence
  Synthetic construct
GAKKSKKRF

SEQ ID NO: 297 - protein, 8 aa, Artificial Sequence
  Synthetic construct
GAKKSKKR

SEQ ID NO: 298 - protein, 7 aa, Artificial Sequence
  Synthetic construct
GAKKSKK

SEQ ID NO: 299 - skipped

SEQ ID NO: 300 - protein, 29 aa, Artificial Sequence
  Synthetic construct
GAKKSKKRFSFKKSFKLSGFSFKKNKKEA

SEQ ID NO: 301 - protein, 29 aa, Artificial Sequence
  Synthetic construct
GAKKAKKRFSFKKSFKLSGFSFKKNKKEA

SEQ ID NO: 302 - protein, 28 aa, Artificial Sequence
  Synthetic construct
GAQESKKKKKKRFSFKKSFKLSGFSFKK

SEQ ID NO: 303 - protein, 27 aa, Artificial Sequence
  Synthetic construct
GAQESKKKKKKRFSFKKSFKLSGFSFK

SEQ ID NO: 304 - protein, 26 aa, Artificial Sequence
  Synthetic construct
GAQESKKKKKKRFSFKKSFKLSGFSF

SEQ ID NO: 305 - protein, 25 aa, Artificial Sequence
  Synthetic construct
GAQESKKKKKKRFSFKKSFKLSGFS

SEQ ID NO: 306 - protein, 24 aa, Artificial Sequence
  Synthetic construct
GAQESKKKKKKRFSFKKSFKLSGF

SEQ ID NO: 307 - protein, 23 aa, Artificial Sequence
  Synthetic construct
GAQESKKKKKKRFSFKKSFKLSG

SEQ ID NO: 308 - protein, 22 aa, Artificial Sequence
  Synthetic construct
GAQESKKKKKKRFSFKKSFKLS

SEQ ID NO: 309 - protein, 21 aa, Artificial Sequence
  Synthetic construct
GAQESKKKKKKRFSFKKSFKL

SEQ ID NO: 310 - protein, 20 aa, Artificial Sequence
  Synthetic construct
GAQESKKKKKKRFSFKKSFK

SEQ ID NO: 311 - protein, 19 aa, Artificial Sequence
  Synthetic construct
GAQESKKKKKKRFSFKKSF

SEQ ID NO: 312 - protein, 18 aa, Artificial Sequence
  Synthetic construct
GAQESKKKKKKRFSFKKS

SEQ ID NO: 313 - protein, 17 aa, Artificial Sequence
  Synthetic construct
GAQESKKKKKKRFSFKK

SEQ ID NO: 314 - protein, 16 aa, Artificial Sequence
  Synthetic construct
GAQESKKKKKKRFSFK

SEQ ID NO: 315 - protein, 15 aa, Artificial Sequence
  Synthetic construct
GAQESKKKKKKRFSF

SEQ ID NO: 316 - protein, 14 aa, Artificial Sequence
  Synthetic construct
GAQESKKKKKKRFS

SEQ ID NO: 317 - protein, 13 aa, Artificial Sequence
  Synthetic construct
GAQESKKKKKKRF

SEQ ID NO: 318 - protein, 12 aa, Artificial Sequence
  Synthetic construct
GAQESKKKKKKR

SEQ ID NO: 319 - protein, 11 aa, Artificial Sequence
  Synthetic construct
GAQESKKKKKK

SEQ ID NO: 320 - protein, 10 aa, Artificial Sequence
  Synthetic construct
GAQESKKKKK

SEQ ID NO: 321 - protein, 9 aa, Artificial Sequence
  Synthetic construct
GAQESKKKK

SEQ ID NO: 322 - protein, 8 aa, Artificial Sequence
  Synthetic construct
GAQESKKK

SEQ ID NO: 323 - protein, 7 aa, Artificial Sequence
  Synthetic construct
GAQESKK

SEQ ID NO: 324 - protein, 30 aa, Artificial Sequence
  Synthetic construct
GSQSSKKKKKKFSFKKPFKLSGLSFKRNRK

SEQ ID NO: 325 - protein, 29 aa, Artificial Sequence
  Synthetic construct
GSQSSKKKKKKFSFKKPFKLSGLSFKRNR

SEQ ID NO: 326 - protein, 28 aa, Artificial Sequence
  Synthetic construct
GSQSSKKKKKKFSFKKPFKLSGLSFKRN

SEQ ID NO: 327 - protein, 27 aa, Artificial Sequence
  Synthetic construct
GSQSSKKKKKKFSFKKPFKLSGLSFKR

SEQ ID NO: 328 - protein, 26 aa, Artificial Sequence
  Synthetic construct
GSQSSKKKKKKFSFKKPFKLSGLSFK

SEQ ID NO: 329 - protein, 25 aa, Artificial Sequence
  Synthetic construct
GSQSSKKKKKKFSFKKPFKLSGLSF

SEQ ID NO: 330 - protein, 24 aa, Artificial Sequence
  Synthetic construct
GSQSSKKKKKKFSFKKPFKLSGLS

SEQ ID NO: 331 - protein, 23 aa, Artificial Sequence
  Synthetic construct
GSQSSKKKKKKFSFKKPFKLSGL

SEQ ID NO: 332 - protein, 22 aa, Artificial Sequence
  Synthetic construct
GSQSSKKKKKKFSFKKPFKLSG

SEQ ID NO: 333 - protein, 21 aa, Artificial Sequence
  Synthetic construct
GSQSSKKKKKKFSFKKPFKLS

SEQ ID NO: 334 - protein, 20 aa, Artificial Sequence
  Synthetic construct
GSQSSKKKKKKFSFKKPFKL

SEQ ID NO: 335 - protein, 19 aa, Artificial Sequence
  Synthetic construct
GSQSSKKKKKKFSFKKPFK

SEQ ID NO: 336 - protein, 18 aa, Artificial Sequence
  Synthetic construct
GSQSSKKKKKKFSFKKPF

SEQ ID NO: 337 - protein, 17 aa, Artificial Sequence
  Synthetic construct
GSQSSKKKKKKFSFKKP

SEQ ID NO: 338 - protein, 16 aa, Artificial Sequence
  Synthetic construct
GSQSSKKKKKKFSFKK

SEQ ID NO: 339 - protein, 15 aa, Artificial Sequence
  Synthetic construct
GSQSSKKKKKKFSFK

SEQ ID NO: 340 - protein, 14 aa, Artificial Sequence
  Synthetic construct
GSQSSKKKKKKFSF

SEQ ID NO: 341 - protein, 13 aa, Artificial Sequence
  Synthetic construct
GSQSSKKKKKKFS

SEQ ID NO: 342 - protein, 12 aa, Artificial Sequence
  Synthetic construct
GSQSSKKKKKKF

SEQ ID NO: 343 - protein, 11 aa, Artificial Sequence
  Synthetic construct
GSQSSKKKKKK

SEQ ID NO: 344 - protein, 10 aa, Artificial Sequence
  Synthetic construct
GSQSSKKKKK

SEQ ID NO: 345 - protein, 9 aa, Artificial Sequence
  Synthetic construct
GSQSSKKKK

SEQ ID NO: 346 - protein, 8 aa, Artificial Sequence
  Synthetic construct
GSQSSKKK

SEQ ID NO: 347 - protein, 7 aa, Artificial Sequence
  Synthetic construct
GSQSSKK

SEQ ID NO: 348 - protein, 28 aa, Artificial Sequence
  Synthetic construct
GAKKAKKRFSFKKSFKLSGFSFKKNKKE

SEQ ID NO: 349 - protein, 27 aa, Artificial Sequence
  Synthetic construct
GAKKAKKRFSFKKSFKLSGFSFKKNKK

SEQ ID NO: 350 - protein, 26 aa, Artificial Sequence
  Synthetic construct
GAKKAKKRFSFKKSFKLSGFSFKKNK

SEQ ID NO: 351 - protein, 25 aa, Artificial Sequence
  Synthetic construct
GAKKAKKRFSFKKSFKLSGFSFKKN

SEQ ID NO: 352 - protein, 24 aa, Artificial Sequence
  Synthetic construct
GAKKAKKRFSFKKSFKLSGFSFKK

SEQ ID NO: 353 - protein, 23 aa, Artificial Sequence
  Synthetic construct
GAKKAKKRFSFKKSFKLSGFSFK

SEQ ID NO: 354 - protein, 22 aa, Artificial Sequence
  Synthetic construct
GAKKAKKRFSFKKSFKLSGFSF

SEQ ID NO: 355 - protein, 21 aa, Artificial Sequence
  Synthetic construct
GAKKAKKRFSFKKSFKLSGFS

SEQ ID NO: 356 - protein, 20 aa, Artificial Sequence
  Synthetic construct
GAKKAKKRFSFKKSFKLSGF

SEQ ID NO: 357 - protein, 19 aa, Artificial Sequence
  Synthetic construct
GAKKAKKRFSFKKSFKLSG

SEQ ID NO: 358 - protein, 18 aa, Artificial Sequence
  Synthetic construct
GAKKAKKRFSFKKSFKLS

SEQ ID NO: 359 - protein, 17 aa, Artificial Sequence
  Synthetic construct
GAKKAKKRFSFKKSFKL

SEQ ID NO: 360 - protein, 16 aa, Artificial Sequence
  Synthetic construct
GAKKAKKRFSFKKSFK

SEQ ID NO: 361 - protein, 15 aa, Artificial Sequence
  Synthetic construct
GAKKAKKRFSFKKSF

SEQ ID NO: 362 - protein, 14 aa, Artificial Sequence
  Synthetic construct
GAKKAKKRFSFKKS

SEQ ID NO: 363 - protein, 13 aa, Artificial Sequence
  Synthetic construct
GAKKAKKRFSFKK

SEQ ID NO: 364 - protein, 12 aa, Artificial Sequence
  Synthetic construct
GAKKAKKRFSFK

SEQ ID NO: 365 - protein, 11 aa, Artificial Sequence
  Synthetic construct
GAKKAKKRFSF

SEQ ID NO: 366 - protein, 10 aa, Artificial Sequence
  Synthetic construct
GAKKAKKRFS

SEQ ID NO: 367 - protein, 9 aa, Artificial Sequence
  Synthetic construct
GAKKAKKRF

SEQ ID NO: 368 - protein, 8 aa, Artificial Sequence
  Synthetic construct
GAKKAKKR

SEQ ID NO: 369 - protein, 7 aa, Artificial Sequence
  Synthetic construct
GAKKAKK

SEQ ID NO: 370 - protein, 95 aa, Artificial Sequence
  Synthetic construct
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 371 - protein, 96 aa, Artificial Sequence
  Synthetic construct
MSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 372 - protein, 94 aa, Artificial Sequence
  Synthetic construct
MTEQQWNFAGIEAAASAIQGNVTSIHSLDEGKQSLTKLAAAWGGSGSEAYQGVQQKWDATATELNNALQNLARTISEAGQAMASTEGNVTGMFA

SEQ ID NO: 373 - protein, 8 aa, Artificial Sequence
  Synthetic construct
  MOD_RES (2)..(2): X is any amino acid
GXKLSKKK

(19) United States
(12) Patent Application Publication
(10) Pub. No.: US 2022/0218811 A1
(43) Pub. Date: Jul. 14, 2022

(54) METHODS OF TREATING TUBERCULOSIS

(71) Applicant: Codiak BioSciences, Inc., Cambridge, MA (US)

(72) Inventors: Kyriakos ECONOMIDES, Grafton, MA (US); Nuruddeen LEWIS, Andover, MA (US)

(73) Assignee: Codiak BioSciences, Inc., Cambridge, MA (US)

(21) Appl. No.: 17/594,401

(22) PCT Filed: Apr. 17, 2020
(86) PCT No.: PCT/US2020/028778

§ 371 Date: Oct. 14, 2021

Related U.S. Application Data
(60) Provisional application No. 62/851,567, filed on May 22, 2019.
(60) Provisional application No. 62/840,330, filed on Apr. 29, 2019.
(60) Provisional application No. 62/835,427, filed on Apr. 17, 2019.

(51) Int. Cl. A61K 39/04; A61K 9/127; A61K 38/20; A61P 31/06

(52) U.S. Cl. CPC A61K 39/04; A61K 9/127; A61K 2039/6018; A61P 31/06; A61K 38/208

(57) ABSTRACT

The present disclosure relates to extracellular vesicles, e.g., exosomes, comprising a cytokine, e.g., Interleukin-12 (IL-12), and, optionally, a TB antigen. Also provided herein are methods of inducing an immune response against Mycobacterium tuberculosis in a subject in need thereof comprising administering an extracellular vesicle comprising a cytokine, e.g., IL-12, and, optionally, a TB antigen.

### CROSS-REFERENCE TO RELATED APPLICATIONS

This PCT application claims the priority benefit of U.S. Provisional Application Nos. 62/835,427 filed on Apr. 17, 2019; 62/840,330 filed on Apr. 29, 2019; and 62/851,567 filed on May 22, 2019, each of which is incorporated herein by reference in its entirety.

### REFERENCE TO SEQUENCE LISTING SUBMITTED ELECTRONICALLY VIA EFS-WEB

The content of the electronically submitted sequence listing (Name: 4000_0430000_SL_ST25.txt, Size: 88,935 bytes; and Date of Creation: Apr. 17, 2020) submitted in this application is incorporated herein by reference in its entirety.

### FIELD OF DISCLOSURE

The present disclosure relates to modified extracellular vesicles, e.g., exosomes, (e.g., comprising IL-12) that can be used to treat and/or prevent Mycobacterium tuberculosis infection (“TB”). The present disclosure also relates to methods of producing such EVs, exosomes, and uses thereof.

### BACKGROUND

Tuberculosis (TB) remains a significant cause of death around the world. The World Health Organization (WHO) estimates that in 2017 as many as 10 million people were infected with TB worldwide, leading to an estimated 1.3 million deaths. Though treatments exist that allow many patients to recover, the United States Center for Disease Control noted in 2017 that the rate of decline in new cases of TB in the U.S. remains too slow to achieve the elimination of TB in the U.S. this century. Accordingly, there remains a strong need to improve on and develop novel means of treating TB.

EVs, exosomes, are important mediators of intercellular communication. They are also important biomarkers in the diagnosis and prognosis of many diseases. As drug delivery vehicles, EVs, e.g., exosomes offer many advantages over traditional drug delivery methods (e.g., peptide immunization, DNA vaccines) as a new treatment modality in many therapeutic areas.

### SUMMARY OF DISCLOSURE

Certain aspects of the present disclosure are directed to a method of inducing an immune response against Mycobacterium tuberculosis in a subject in need thereof comprising administering extracellular vesicles (EVs) with Interleukin-12 (IL-12) expressed on their surface.

In some aspects, the immune response is against one or more epitopes of Mycobacterium tuberculosis. In some aspects, the immune response is a cellular immune response, a humoral immune response, or both cellular and humoral immune responses. In some aspects, the induction of the cellular immune response, the humoral immune response, or both cellular and humor immune responses of the EV is increased by at least about 5%, at least about 10%, at least about 20%, at least about 30%, at least about 40%, at least about 50%, at least about 60%, at least about 70%, at least about 80%, at least about 90%, at least about 100% or more, compared to IL-12 in a free form not expressed on EVs.

In some aspects, the immune response is a CD4 T cell response, a CD8 T cell response, or both CD4 and CD8 T cell responses. In some aspects, the epitope is an ESAT6 antigen, a TB10.4 antigen, or both ESAT6 antigen and TB10.4 antigen.

In some aspects, the immune response is CD4 T-cell immune response with effector function that is specific to the ESAT6 antigen. In some aspects, the immune response is CD8 T-cell immune response that is specific to the TB10.4 antigen.

In some aspects, the ESAT6 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in

(GenBank: AWM98862.1; SEQ ID NO: 370)
MTEQQWNFAGIEAAASAIQGNVTSIHSLLDEGKQSLTKLAAAWGGSGS
EAYQGVQQKWDATATELNNALQNLARTISEAGQAMASTEGNVTGMFA.

In some aspects, the TB10.4 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in

(GenBank: ANZ80905.1; SEQ ID NO: 371)
MMSQIMYNYPAMLGHAGDMAGYAGTLQSLGAEIAVEQAALQSAWQGDTG
ITYQAWQAQWNQAMEDLVRAYHAMSSTHEANTMAMMARDTAEAAKWGG.

In some aspects, the EV further comprises a TB antigen.

Certain aspects of the present disclosure are directed to an extracellular vesicle (EV) comprising IL-12 and a TB antigen. In some aspects, the EV induces an immune response against Mycobacterium tuberculosis in a subject in need thereof. In some aspects, the immune response is against one or more epitopes of Mycobacterium tuberculosis.

In some aspects, the immune response is a cellular immune response, a humoral immune response, or both cellular and humoral immune responses. In some aspects, the induction of the cellular immune response, the humoral immune response, or both cellular and humor immune responses of the EV is increased by at least about 5%, at least about 10%, at least about 20%, at least about 30%, at least about 40%, at least about 50%, at least about 60%, at least about 70%, at least about 80%, at least about 90%, at least about 100% or more, compared to IL-12 in a free form not expressed on EVs. In some aspects, the immune response is a CD4 T cell response, a CD8 T cell response, or both CD4 and CD8 T cell responses. In some aspects, the epitope is an ESAT6 antigen, a TB10.4 antigen, or both ESAT6 antigen and TB10.4 antigen.

In some aspects, the immune response is CD4 T-cell immune response with effector function that is specific to the ESAT6 antigen. In some aspects, the immune response is CD8 T-cell immune response that is specific to the TB10.4 antigen.

In some aspects, the ESAT6 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in MTEQQWNFAGIEAAASAIQGNVTSIHS LLDEGKQSLTKLAAAWGGSGSEAYQGVQQKWDATATELNNALQNLARTISEAGQAMASTEGNVTGMFA (NCBI Reference Sequence: WP_053892578.1; SEQ ID NO: 370). In other aspects, the ESAT6 antigen comprises MTEQQWNFAGIEAAASAIQGNVTS IHS LLDEGKQSLTKLAAAWGGSGSEAYQGVQQKWDATATELNNALQNLARTISEAGQAMASTEGNVTGMFA (SEQ ID NO: 370).

In some aspects, the TB10.4 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in

(SEQ ID NO: 371)
MSQIMYNYPAMLGHAGDMAGYAGTLQSLGAEIAVEQAALQSAWQGDTGI
TYQAWQAQWNQAMEDLVRAYHAMSSTHEANTMAMMARDTAEAAKWGG.

In other aspects, the TB10.4 antigen comprises

(SEQ ID NO: 371)
MSQIMYNYPAMLGHAGDMAGYAGTLQSLGAEIAVEQAALQSAWQGDTGI
TYQAWQAQWNQAMEDLVRAYHAMSSTHEANTMAMMARDTAEAAKWGG.

In some aspects, the TB antigen is identical to the ESAT6 antigen or the TB10.4 antigen.

In some aspects, the EV further comprises a scaffold moiety. In some aspects, the IL-12 is linked to the scaffold moiety. In some aspects, the TB antigen is further linked to the scaffold moiety. In some aspects, the EV further comprises a second scaffold moiety. In some aspects, the IL-12 is linked to the scaffold moiety, and the TB antigen is linked to the second scaffold moiety.

In some aspects, the first scaffold moiety and the second scaffold moiety are the same. In some aspects, the first scaffold moiety and the second scaffold moiety are different.

In some aspects, the first scaffold moiety is a Scaffold X. In some aspects, the first scaffold moiety is a Scaffold Y. In some aspects, the second scaffold moiety is a Scaffold Y.

In some aspects, the second scaffold moiety is a Scaffold X. In some aspects, Scaffold X is a scaffold protein that is capable of anchoring the IL-12 and/or the TB antigen on the luminal surface of the EV and/or on the exterior surface of the EV.

In some aspects, Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), and any combination thereof. In some aspects, the first scaffold moiety is PTGFRN protein. In some aspects, the first scaffold moiety comprises an amino acid sequence as set forth in SEQ ID NO: 33. In some aspects, the first scaffold moiety comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 1.

In some aspects, Scaffold Y is a scaffold protein that is capable of anchoring the IL-12 and/or the TB antigen on the luminal surface of the EV. In some aspects, the Scaffold Y is selected from the group consisting of myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), and any combination thereof. In some aspects, the Scaffold Y is BASP1 protein.

In some aspects, the Scaffold Y comprises an N terminus domain (ND) and an effector domain (ED), wherein the ND and/or the ED are associated with the luminal surface of the EV. In some aspects, the ND is associated with the luminal surface of the exosome via myristoylation. In some aspects, the ED is associated with the luminal surface of the exosome by an ionic interaction.

In some aspects, the ED comprises (i) a basic amino acid or (ii) two or more basic amino acids in sequence, wherein the basic amino acid is selected from the group consisting of Lys, Arg, His, and any combination thereof. In some aspects, the basic amino acid is (Lys)n, wherein n is an integer between 1 and 10. In some aspects, the ED comprises Lys (K), KK, KKK, KKKK (SEQ ID NO: 205), KKKKK (SEQ ID NO: 206), Arg (R), RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof.

In some aspects, the ND comprises the amino acid sequence as set forth in G:X2:X3:X4:X5:X6, wherein G represents Gly; wherein “:” represents a peptide bond, wherein each of the X2 to the X6 is independently an amino acid, and wherein the X6 comprises a basic amino acid. In some aspects, (i) the X2 is selected from the group consisting of Pro, Gly, Ala, and Ser; (ii) the X4 is selected from the group consisting of Pro, Gly, Ala, Ser, Val, Ile, Leu, Phe, Trp, Tyr, Gln and Met; (iii) the X5 is selected from the group consisting of Pro, Gly, Ala, and Ser; (iv) the X6 is selected from the group consisting of Lys, Arg, and His; or (v) any combination of (i)-(iv).

In some aspects, the ND comprises the amino acid sequence of G:X2:X3:X4:X5:X6, wherein (i) G represents Gly; (ii) “:” represents a peptide bond; (iii) the X2 is an amino acid selected from the group consisting of Pro, Gly, Ala, and Ser; (iv) the X3 is an amino acid; (v) the X4 is an amino acid selected from the group consisting of Pro, Gly, Ala, Ser, Val, Ile, Leu, Phe, Trp, Tyr, Gln and Met; (vi) the X5 is an amino acid selected from the group consisting of Pro, Gly, Ala, and Ser; and (vii) the X6 is an amino acid selected from the group consisting of Lys, Arg, and His. In some aspects, the X3 is selected from the group consisting of Asn, Gln, Ser, Thr, Asp, Glu, Lys, His, and Arg.

In some aspects, the ND and the ED are joined by a linker. In some aspects, the linker useful for the present disclosure comprises one or more amino acids.

In some aspects, the ND comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), (v) GGKLSKK (SEQ ID NO: 211), or (vi) any combination thereof. In some aspects, the ND comprises an amino acid sequence selected from the group consisting of (i) GGKLSKKK (SEQ ID NO: 238), (ii) GGKLSKKS (SEQ ID NO: 239), (iii) GAKLSKKK (SEQ ID NO: 240), (iv) GAKLSKKS (SEQ ID NO: 241), (v) GGKQSKKK (SEQ ID NO: 242), (vi) GGKQSKKS (SEQ ID NO: 243), (vii) GGKLAKKK (SEQ ID NO: 244), (viii) GGKLAKKS (SEQ ID NO: 245), (ix) GGKLSKKK (SEQ ID NO: 238), (x) GGKLSKKS (SEQ ID NO: 239), and (xi) any combination thereof. In some aspects, the ND comprises the amino acid sequence GGKLSKK (SEQ ID NO: 211).

In some aspects, the Scaffold Y is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 120, at least about 130, at least about 140, at least about 150, at least about 160, at least about 170, at least about 180, at least about 190, or at least about 200 amino acids in length.

In some aspects, the Scaffold Y comprises (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 291).

In some aspects, the Scaffold Y consists of (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 291).

In some aspects, the Scaffold Y does not comprise Met at the N terminus. In some aspects, the Scaffold Y comprises a myristoylated amino acid residue at the N terminus of the scaffold protein. In some aspects, the amino acid residue at the N terminus of the Scaffold Y is Gly.

In some aspects, the IL-12 is linked to a scaffold moiety (e.g., Scaffold X) on the exterior surface of the EV, and the TB antigen is linked to the scaffold moiety on the luminal surface of the EV. In some aspects, the IL-12 is linked to a scaffold moiety (e.g., Scaffold X) on the luminal surface of the EV, and the TB antigen is linked to the scaffold moiety on the exterior surface of the EV. In some aspects, the TB antigen is linked to a scaffold moiety (e.g., Scaffold X) on the exterior surface of the EV, and the IL-12 is linked to a second scaffold moiety (e.g., Scaffold X) on the exterior surface of the EV. In some aspects, the TB antigen is linked to a scaffold moiety (e.g., Scaffold X) on the exterior surface of the EV, and the IL-12 is linked to a second scaffold moiety (e.g., Scaffold Y) on the luminal surface of the EV. In some aspects, the IL-12 is linked to a scaffold moiety (e.g., Scaffold X) on the exterior surface of the EV, and the TB antigen is linked to a second scaffold moiety (e.g., Scaffold X) on the exterior surface of the EV. In some aspects, the IL-12 is linked to a scaffold moiety (e.g., Scaffold X) on the exterior surface of the EV, and the TB antigen is linked to a second scaffold moiety (e.g., Scaffold Y) on the luminal surface of the EV. In some aspects, the IL-12 is linked to a scaffold moiety (e.g., Scaffold Y) on the luminal surface of the EV, and the TB antigen is linked to a second scaffold moiety (e.g., Scaffold Y) on the luminal surface of the EV. In some aspects, the IL-12 is linked to a scaffold moiety (e.g., Scaffold X) on the luminal surface of the EV, and the TB antigen is linked to a second scaffold moiety (e.g., Scaffold Y) on the luminal surface of the EV.

In some aspects, the IL-12 and/or the TB antigen is linked to the scaffold moiety by a linker. In some aspects, the IL-12 and/or the TB antigen is linked to the second scaffold moiety by a linker. In some aspects, the linker is a polypeptide. In some aspects, the linker is a non-polypeptide moiety.

In some aspects, the EV is an exosome.

Certain aspects of the present disclosure are directed to a pharmaceutical composition comprising an EV disclosed herein and a pharmaceutically acceptable carrier.

Certain aspects of the present disclosure are directed to a cell that produces an EV disclosed herein.

Certain aspects of the present disclosure are directed to a cell comprising one or more vectors, wherein the vectors comprise a nucleic acid sequence encoding the TB antigen disclosed herein or IL-12.

Certain aspects of the present disclosure are directed to a kit comprising an EV disclosed herein and instructions for use.

Certain aspects of the present disclosure are directed to a method of making EVs comprising culturing a cell described herein under a suitable condition and obtaining the EVs.

Certain aspects of the present disclosure are directed to a method of inducing an immune response in a subject in need thereof comprising administering an EV disclosed herein to the subject.

Certain aspects of the present disclosure are directed to a method of preventing or treating a disease in a subject in need thereof, comprising administering an EV disclosed herein, wherein the disease is associated with the TB antigen. In some aspects, the disease is tuberculosis.

In some aspects, the method further comprises administering a TB antigen to the subject. In other aspects, the method further comprises administering an EV comprising a TB antigen to the subject. In some aspects, the TB antigen or the EV comprising a TB antigen is administered prior to, concurrently with, or after the administration of the EV. In some aspects, the subject is primed by the TB antigen. In some aspects, the EV is administered parenterally, orally, intravenously, intramuscularly, intra-tumorally, intranasally, subcutaneously, or intraperitoneally.

In some aspects, the method further comprises administering an additional therapeutic agent.

### BRIEF DESCRIPTION OF FIGURES

FIGS. 1A-1B are confocal microscopy images of alveolar tissue following delivery of exosomes carrying a GFP marker. Small arrows label macrophages, pneumocytes, and endothelial cells, each of which shows a positive GFP signal.

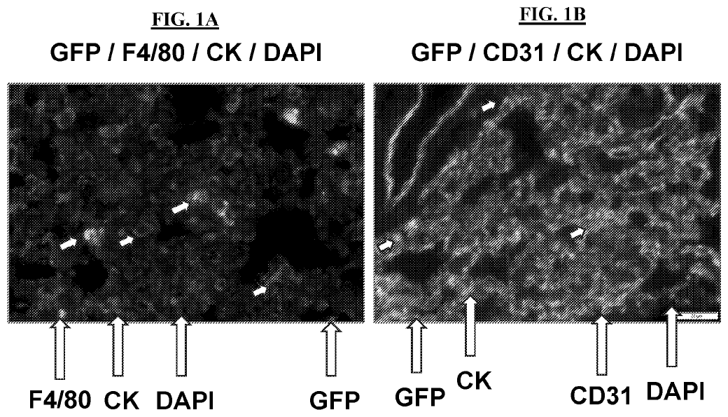

FIG. 2 is a schematic representation of a dosing schedule for measuring the effect of EV-delivered IL-12 in a TB mouse model.

FIGS. 3A-3B are graphical representations of the number of ESAT6⁺ cells (FIG. 3A) and the number of TB10.4⁺ cells (FIG. 3B) in TB mice following administration of isoniazid, carrier control EV (“EXOs”), recombinant IL-12 (“rIL-12”), and EVs loaded with IL-12 (“exoIL-12”). Significance was measured using a one-way ANOVA test; * P<0.005; P<0.0001.

FIGS. 4A-4C are graphical representations of the number of CD4⁺ cells also expressing interferon gamma (IFNγ) and tumor necrosis factor alpha (TNFα) (FIG. 4A), IFNγ and interleukin 2 (IL2) (FIG. 4B), or IFNγ and not IL2 (FIG. 4C), following stimulation with ESAT6 antigen. Significance was measured using a one-way ANOVA test; * P<0.05; ** P<0.01; *** P<0.001; **** P<0.0001.

FIGS. 5A-5C are graphical representations of the number of CD8⁺ cells also expressing IFNγ and TNFα (FIG. 5A), IFNγ and IL2 (FIG. 5B), or IFNγ and not IL2 (FIG. 5C), following stimulation with TB10.4 peptide. Significance was measured using a one-way ANOVA test; * P<0.05; ** P<0.01; *** P<0.001; **** P<0.0001.

FIG. 6 is a schematic representation of a prophylactic dosing schedule for measuring the effect of EV-delivered IL-12 in a TB mouse model.

### DETAILED DESCRIPTION OF DISCLOSURE

The present disclosure is directed to methods of inducing an immune response against Mycobacterium tuberculosis in a subject in need thereof, comprising administering an EV, e.g., exosome, comprising a cytokine, e.g., IL-12, to the subject. In some aspects, the cytokine, e.g., IL-12, is attached (or linked) to one or more scaffold moieties on the surface of the EV, e.g., exosome, or on the luminal surface of the EV, e.g., exosome. In some aspects, the EV further comprises a TB antigen.

Some aspects of the present disclosure are directed to methods of inducing an immune response against Mycobacterium tuberculosis in a subject in need thereof, comprising administering an EV, e.g., exosome, comprising an IL-12 and a TB antigen.

Non-limiting examples of the various aspects are shown in the present disclosure.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vehicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In certain aspects, an exosome comprises a scaffold moiety. As described infra, exosome can be derived from a producer cell, and isolated from the producer cell based on its size, density, biochemical parameters, or a combination thereof. In some aspects, the EVs, e.g., exosomes, of the present disclosure are produced by cells that express one or more transgene products.

As used herein, the term “nanovesicle” refers to an extracellular vesicle with a diameter between 20-250 nm (e.g., between 30-150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In certain aspects, a nanovesicle comprises a scaffold moiety. Nanovesicles, once derived from a producer cell, can be isolated from the producer cell based on its size, density, biochemical parameters, or a combination thereof.

As used herein the term “surface-engineered EVs, e.g., exosomes” (e.g., Scaffold X-engineered EVs, e.g., exosomes) refers to an EV, e.g., exosome, with the membrane or the surface of the EV, e.g., exosome, modified in its composition so that the surface of the engineered EV, e.g., exosome, is different from that of the EV, e.g., exosome, prior to the modification or of the naturally occurring EV, e.g., exosome. The engineering can be on the surface of the EV, e.g., exosome, or in the membrane of the EV, e.g., exosome, so that the surface of the EV, e.g., exosome, is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, e.g., exosome, comprises an exogenous protein (i.e., a protein that the EV, e.g., exosome, does not naturally express) or a fragment or variant thereof that can be exposed to the surface of the EV, e.g., exosome, or can be an anchoring point (attachment) for a moiety exposed on the surface of the EV, e.g., exosome. In other aspects, a surface-engineered EV, e.g., exosome, comprises a higher expression (e.g., higher number) of a natural exosome protein (e.g., Scaffold X) or a fragment or variant thereof that can be exposed to the surface of the EV, e.g., exosome, or can be an anchoring point (attachment) for a moiety exposed on the surface of the EV, e.g., exosome.

As used herein the term “lumen-engineered exosome” (e.g., Scaffold Y-engineered exosome) refers to an EV, e.g., exosome, with the membrane or the lumen of the EV, e.g., exosome, modified in its composition so that the lumen of the engineered EV, e.g., exosome, is different from that of the EV, e.g., exosome, prior to the modification or of the naturally occurring EV, e.g., exosome. The engineering can be directly in the lumen or in the membrane of the EV, e.g., exosome so that the lumen of the EV, e.g., exosome is changed. For example, the membrane is modified in its composition of a protein, a lipid, a small molecule, a carbohydrate, etc. so that the lumen of the EV, e.g., exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises an exogenous protein (i.e., a protein that the EV, e.g., exosome does not naturally express) or a fragment or variant thereof that can be exposed in the lumen of the EV, e.g., exosome or can be an anchoring point (attachment) for a moiety exposed on the inner layer of the EV, e.g., exosome. In other aspects, a lumen-engineered EV, e.g., exosome, comprises a higher expression of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVs, e.g., exosomes described herein, refers to an alteration or engineering of an EV, e.g., exosome and/or its producer cell, such that the modified EV, e.g., exosome is different from a naturally-occurring EV, e.g., exosome. In some aspects, a modified EV, e.g., exosome described herein comprises a membrane that differs in composition of a protein, a lipid, a small molecular, a carbohydrate, etc. compared to the membrane of a naturally-occurring EV, e.g., exosome (e.g., membrane comprises higher density or number of natural exosome proteins and/or membrane comprises proteins that are not naturally found in exosomes (e.g., IL-12 and/or a TB antigen). In certain aspects, such modifications to the membrane changes the exterior surface of the EV, e.g., exosome (e.g., surface-engineered EVs, e.g., exosomes described herein). In certain aspects, such modifications to the membrane changes the lumen of the EV, e.g., exosome (e.g., lumen-engineered EVs, e.g., exosomes described herein).

As used herein, the term “scaffold moiety” refers to a molecule that can be used to anchor a payload or any other compound of interest (e.g., IL-12 and/or a TB antigen) to the EV, e.g., exosome either on the luminal surface or on the exterior surface of the EV, e.g., exosome. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV, e.g., exosome. In some aspects, a scaffold moiety comprises a lipid, carbohydrate, or protein that does not naturally exist in the EV, e.g., exosome. In certain aspects, a scaffold moiety is Scaffold X. In some aspects, a scaffold moiety is Scaffold Y. In further aspects, a scaffold moiety comprises both Scaffold X and Scaffold Y. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin (MFGE8), LAMP2, and LAMP2B.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor a moiety (e.g., IL-12 and/or a TB antigen) to the external surface or the luminal surface of the exosome.

As used herein, the term “Scaffold Y” refers to exosome proteins that were newly identified within the lumen of exosomes. See, e.g., International Appl. No. PCT/US2018/061679, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety to the luminal surface of the exosome). In some aspects, a Scaffold Y can anchor a moiety (e.g., IL-12 and/or a TB antigen) to the luminal surface of the EV, e.g., exosome.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV, e.g., exosome. Similarly, in certain aspects, a functional fragment of a Scaffold Y protein retains the ability to anchor a moiety on the luminal surface of the EV, e.g., exosome. Whether a fragment is a functional fragment can be assessed by any art known methods to determine the protein content of EVs, e.g., exosomes including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, antigen, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof. In some aspects variants or variants of fragments of BSG share at least about 70%, at least about 80%, at least about 85%, at least about 90%, at least about 95%, at least about 96%, at least about 97%, at least about 98%, or at least about 99% sequence identity with BSG according to SEQ ID NO: 9 or with a functional fragment thereof. In some aspects variants or variants of fragments of IGSF2 share at least about 70%, at least about 80%, at least about 85%, at least about 90%, at least about 95%, at least about 96%, at least about 97%, at least about 98%, or at least about 99% sequence identity with IGSF2 according to SEQ ID NO: 34 or with a functional fragment thereof. In some aspects variants or variants of fragments of IGSF3 share at least about 70%, at least about 80%, at least about 85%, at least about 90%, at least about 95%, at least about 96%, at least about 97%, at least about 98%, or at least about 99% sequence identity with IGSF3 according to SEQ ID NO: 20 or with a functional fragment thereof. In some aspects variants or variants of fragments of IGSF8 share at least about 70%, at least about 80%, at least about 85%, at least about 90%, at least about 95%, at least about 96%, at least about 97%, at least about 98%, or at least about 99% sequence identity with IGSF8 according to SEQ ID NO: 14 or with a functional fragment thereof. In some aspects variants or variants of fragments of ITGB1 share at least about 70%, at least about 80%, at least about 85%, at least about 90%, at least about 95%, at least about 96%, at least about 97%, at least about 98%, or at least about 99% sequence identity with ITGB1 according to SEQ ID NO: 21 or with a functional fragment thereof. In some aspects variants or variants of fragments of ITGA4 share at least about 70%, at least about 80%, at least about 85%, at least about 90%, at least about 95%, at least about 96%, at least about 97%, at least about 98%, or at least about 99% sequence identity with ITGA4 according to SEQ ID NO: 22 or with a functional fragment thereof. In some aspects variants or variants of fragments of SLC3A2 share at least about 70%, at least about 80%, at least about 85%, at least about 90%, at least about 95%, at least about 96%, at least about 97%, at least about 98%, or at least about 99% sequence identity with SLC3A2 according to SEQ ID NO: 23 or with a functional fragment thereof. In some aspects variants or variants of fragments of ATP1A1 share at least about 70%, at least about 80%, at least about 85%, at least about 90%, at least about 95%, at least about 96%, at least about 97%, at least about 98%, or at least about 99% sequence identity with ATP1A1 according to SEQ ID NO: 24 or with a functional fragment thereof. In some aspects variants or variants of fragments of ATP1A2 share at least about 70%, at least about 80%, at least about 85%, at least about 90%, at least about 95%, at least about 96%, at least about 97%, at least about 98%, or at least about 99% sequence identity with ATP1A2 according to SEQ ID NO: 25 or with a functional fragment thereof. In some aspects variants or variants of fragments of ATP1A3 share at least about 70%, at least about 80%, at least about 85%, at least about 90%, at least about 95%, at least about 96%, at least about 97%, at least about 98%, or at least about 99% sequence identity with ATP1A3 according to SEQ ID NO: 26 or with a functional fragment thereof. In some aspects variants or variants of fragments of ATPlA4 share at least about 70%, at least about 80%, at least about 85%, at least about 90%, at least about 95%, at least about 96%, at least about 97%, at least about 98%, or at least about 99% sequence identity with ATP1A4 according to SEQ ID NO: 27 or with a functional fragment thereof. In some aspects variants or variants of fragments of ATP1B3 share at least about 70%, at least about 80%, at least about 85%, at least about 90%, at least about 95%, at least about 96%, at least about 97%, at least about 98%, or at least about 99% sequence identity with ATP1B3 according to SEQ ID NO: 28 or with a functional fragment thereof. In some aspects variants or variants of fragments of ATP2B1 share at least about 70%, at least about 80%, at least about 85%, at least about 90%, at least about 95%, at least about 96%, at least about 97%, at least about 98%, or at least about 99% sequence identity with ATP2B1 according to SEQ ID NO: 29 or with a functional fragment thereof. In some aspects variants or variants of fragments of ATP2B2 share at least about 70%, at least about 80%, at least about 85%, at least about 90%, at least about 95%, at least about 96%, at least about 97%, at least about 98%, or at least about 99% sequence identity with ATP2B2 according to SEQ ID NO: 30 or with a functional fragment thereof. In some aspects variants or variants of fragments of ATP2B3 share at least about 70%, at least about 80%, at least about 85%, at least about 90%, at least about 95%, at least about 96%, at least about 97%, at least about 98%, or at least about 99% sequence identity with ATP2B3 according to SEQ ID NO: 31 or with a functional fragment thereof. In some aspects variants or variants of fragments of ATP2B4 share at least about 70%, at least about 80%, at least about 85%, at least about 90%, at least about 95%, at least about 96%, at least about 97%, at least about 98%, or at least about 99% sequence identity with ATP2B4 according to SEQ ID NO: 32 or with a functional fragment thereof. In some aspects, the variant or variant of a fragment of Scaffold X protein disclosed herein retains the ability to be specifically targeted to EVs, e.g., exosomes. In some aspects, the Scaffold X includes one or more mutations, for example, conservative amino acid substitutions.

In some aspects, a variant of a Scaffold Y comprises a variant having at least 70% identity to MARCKS, MARCKSL1, BASP1, or a fragment of MARCKS, MARCKSL1, or BASP1. In some aspects variants or variants of fragments of MARCKS share at least about 70%, at least about 80%, at least about 85%, at least about 90%, at least about 95%, at least about 96%, at least about 97%, at least about 98%, or at least about 99% sequence identity with MARCKS according to SEQ ID NO: 47 or with a functional fragment thereof. In some aspects variants or variants of fragments of MARCKSL1 share at least about 70%, at least about 80%, at least about 85%, at least about 90%, at least about 95%, at least about 96%, at least about 97%, at least about 98%, or at least about 99% sequence identity with MARCKSL1 according to SEQ ID NO: 48 or with a functional fragment thereof. In some aspects variants or variants of fragments of BASP1 share at least about 70%, at least about 80%, at least about 85%, at least about 90%, at least about 95%, at least about 96%, at least about 97%, at least about 98%, or at least about 99% sequence identity with BASP1 according to SEQ ID NO: 49 or with a functional fragment thereof. In some aspects, the variant or variant of a fragment of Scaffold Y protein retains the ability to be specifically targeted to the luminal surface of EVs, e.g., exosomes. In some aspects, the Scaffold Y includes one or more mutations, e.g., conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity may be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-la. They used random mutagenesis to generate over 3,500 individual IL-la mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the term “linked to” or “conjugated to” are used interchangeably and refer to a covalent or non-covalent bond formed between a first moiety and a second moiety, e.g., Scaffold X and IL-12 or a TB antigen, respectively, e.g., a scaffold moiety expressed in or on the extracellular vesicle and IL-12 or a TB antigen, e.g., Scaffold X (e.g., a PTGFRN protein), respectively, in the luminal surface of or on the external surface of the extracellular vesicle.

The term “encapsulated”, or grammatically different forms of the term (e.g., encapsulation, or encapsulating) refers to a status or process of having a first moiety (e.g., IL-12 and/or a TB antigen) inside a second moiety (e.g., an EV, e.g., exosome) without chemically or physically linking the two moieties. In some aspects, the term “encapsulated” can be used interchangeably with “in the lumen of”. Non-limiting examples of encapsulating a first moiety (e.g., IL-12 and/or a TB antigen) into a second moiety (e.g., EVs, e.g., exosomes) are disclosed elsewhere herein.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof. In some aspects, the EVs, e.g., exosomes useful in the present disclosure do not carry an antigen on MHC class I or class II molecule exposed on the surface of the EV, e.g., exosome, but instead can carry an antigen in the lumen of the EV, e.g., exosome or on the surface of the EV, e.g., exosome by attachment to Scaffold X and/or Scaffold Y.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “payload” refers to an agent that acts on a target (e.g., a target cell) that is contacted with the EV. Non-limiting examples of payload that can be included on the EV, e.g., exosome, are a cytokine, e.g., IL-12 and/or a TB antigen. Payloads that can be introduced into an EV, e.g., exosome, and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises a cytokine. In certain aspects, the payload comprises IL-12. As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. As used herein, the term “antigen” refers to any agent that when introduced into a subject elicits an immune response (cellular or humoral) to itself. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs, e.g., exosomes, comprise less than 10% of macromolecules by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, contaminant proteins, lipids, carbohydrates, metabolites, or a combination thereof.

As used herein, the term “conventional exosome protein” means a protein previously known to be enriched in exosomes, including but is not limited to CD9, CD63, CD81, PDGFR, GPI anchor proteins, lactadherin (MFGE8), LAMP2, and LAMP2B, a fragment thereof, or a peptide that binds thereto.

“Administering,” as used herein, means to give a composition comprising an EV, e.g., exosome, disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. EVs, e.g., exosomes can be administered as part of a pharmaceutical composition comprising at least one excipient.

An “immune response,” as used herein, refers to a biological response within a vertebrate against foreign agents or abnormal, e.g., infected cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly, an immune response can comprise a humoral immune response (e.g., mediated by B-cells), cellular immune response (e.g., mediated by T cells), or both humoral and cellular immune responses. In some aspects, an immune response is an “inhibitory” immune response. An inhibitory immune response is an immune response that blocks or diminishes the effects of a stimulus (e.g., antigen). In certain aspects, the inhibitory immune response comprises the production of inhibitory antibodies against the stimulus. In some aspects, the inhibitory response comprises the production of antibodies against the cytokine, e.g., antibodies against IL-12. In some aspects, an immune response is a “stimulatory” immune response. A stimulatory immune response is an immune response that results in the generation of effectors cells (e.g., cytotoxic T lymphocytes) that can destroy and clear a target antigen (e.g., a Mycobacterium tuberculosis antigen).

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also includes prophylaxis or prevention of a disease or condition or its symptoms thereof. In one aspect, the term “treating” or “treatment” means inducing an immune response in a subject against an antigen. In some aspects, the disease or condition is associated with a Mycobacterium tuberculosis infection. In certain aspects, the disease or condition is TB.

“Prevent” or “preventing,” as used herein, refers to decreasing or reducing the occurrence or severity of a particular outcome. In some aspects, preventing an outcome is achieved through prophylactic treatment. In some aspects, an EV, e.g., an exosome, comprising a cytokine, e.g., IL-12, described herein is administered to a subject prophylactically. In some aspects, the subject is at risk of Mycobacterium tuberculosis infection. In some aspects, the subject has been exposed to Mycobacterium tuberculosis. In some aspects, the subject has been exposed to Mycobacterium tuberculosis but does not show signs of infection.

“Tuberculosis” or “TB,” as used herein, refers to a disease caused by Mycobacterium tuberculosis infection. TB generally affects the lungs, though it can also affect the brain, kidneys, or spine of an infected patient. Mycobacterium tuberculosis infection does not necessarily lead to overt symptoms of TB. Rather, there are two forms of Mycobacterium tuberculosis infection: latent TB infection and TB disease. “Latent” TB infection does not present with any symptoms typically associated with TB, and patients having a latent TB infection do not have a contagious form of the infection. Patients having TB disease present with the typical symptoms of TB and have a contagious form of the infection.

### II. Methods of the Disclosure

Certain aspects of the present disclosure are directed to methods of inducing an immune response against Mycobacterium tuberculosis in a subject in need thereof, comprising administering an EV, e.g., an exosome, comprising a cytokine, e.g., IL-12, to the subject. Some aspects of the present disclosure are directed to methods of treating a Mycobacterium tuberculosis infection in a subject in need thereof, comprising administering an EV, e.g., an exosome, comprising a cytokine, e.g., IL-12. Some aspects of the present disclosure are directed to methods of administering an EV, e.g., an exosome, comprising a cytokine, e.g., IL-12, prophylactically to a subject at risk of developing a Mycobacterium tuberculosis infection.

Tuberculosis (TB) remains a significant cause of death around the world. The World Health Organization (WHO) estimates that in 2017, TB caused 1.3 million deaths word-wide out of an estimated 10 million infected individuals, with an additional 300,000 deaths resulting from co-infection with HIV. World Health Organization, Global Tuberculosis Report 2018, p. 1. This places TB infection in the top 10 causes of death worldwide.

Mycobacterium tuberculosis infection typically begins by uptake of Mycobacterium tuberculosis bacterial cells by alveolar macrophages in subject's lungs. About 5-10% of infected subjects then experience active TB disease, wherein the Mycobacterium tuberculosis multiplies, lyses the host cell, and spreads to neighboring host cells resulting in widespread infection and TB disease. In 90-95% of infected subjects, a host immune response leads to suppression and/or eradication of the infection either through apoptosis-mediated cell death, autophagolysosome destruction of the bacterial cells, or phagolysosome destruction of the bacterial cells. The infection cycle of Mycobacterium tuberculosis is shown at Yuk et al., Clin Exp Vaccine Res. 2014 July; 3(2):155-167.

Pro-inflammatory T-helper (Th) type 1 cells are essential for phagocyte activation to promote killing of intracellular Mycobacterium tuberculosis and for chemoattraction of immunocompetent cells to the site of infection. Regulatory and anti-inflammatory responses dampen excessive tissue destruction and play an essential role in the establishment of protection and infection containment within granuloma. Mycobacterium tuberculosis-specific T-cells representing both pro- and anti-inflammatory aspects of infection control are readily detectable in peripheral blood as well. Release of IFNγ is further associated with a cell-mediated immune response, e.g., to Mycobacterium tuberculosis infection. IFNγ release is augmented by APC-derived TNFα and IL-12 and autocrine IFNγ.

Certain aspects of the present disclosure are directed to methods of inducing an immune response against Mycobacterium tuberculosis in a subject in need thereof comprising administering an extracellular vesicle (EV) comprising Interleukin-12 (IL-12). In some aspects, the immune response comprises a cellular immune response. In some aspects, the immune response comprises a humoral immune response. In some aspects, the immune response comprises both a cellular immune response and a humoral immune response.

In some aspects, the induction of the cellular immune response, the humoral immune response, or both cellular and humoral immune responses of the EV is increased by at least about 5%, at least about 10%, at least about 20%, at least about 30%, at least about 40%, at least about 50%, at least about 60%, at least about 70%, at least about 80%, at least about 90%, at least about 100% or more, compared to IL-12 without an EV. In some aspects, the induction of the cellular immune response, the humoral immune response, or both cellular and humoral immune responses of the EV is increased by at least about 5% to at least about 100%, at least about 10% to at least about 100%, at least about 15% to at least about 100%, at least about 20% to at least about 100%, at least about 25% to at least about 100%, at least about 30% to at least about 100%, at least about 40% to at least about 100%, at least about 50% to at least about 100%, at least about 60% to at least about 100%, at least about 70% to at least about 100%, at least about 80% to at least about 100%, or at least about 90% to at least about 100% compared to IL-12 without an EV, i.e., IL-12 in a free form not expressed on EVs.

In some aspects, the induction of the cellular immune response, the humoral immune response, or both cellular and humoral immune responses of the EV is increased by at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 20-fold, at least about 30-fold, at least about 40-fold, at least about 50-fold, at least about 75-fold, or at least about 100-fold compared to IL-12 without an EV. In some aspects, the induction of the cellular immune response, the humoral immune response, or both cellular and humoral immune responses of the EV is increased by at least about 2-fold. In some aspects, the induction of the cellular immune response, the humoral immune response, or both cellular and humoral immune responses of the EV is increased by at least about 3-fold. In some aspects, the induction of the cellular immune response, the humoral immune response, or both cellular and humoral immune responses of the EV is increased by at least about 4-fold. In some aspects, the induction of the cellular immune response, the humoral immune response, or both cellular and humoral immune responses of the EV is increased by at least about 5-fold. In some aspects, the induction of the cellular immune response, the humoral immune response, or both cellular and humoral immune responses of the EV is increased by at least about 10-fold.

In some aspects, the immune response is against one or more epitopes of Mycobacterium tuberculosis. Various antigens are associated with Mycobacterium tuberculosis infection, including ESAT-6, TB10.4, CFP10, Rv2031 (hspX), Rv2654c (TB7.7), and Rv1038c (EsxJ). See, e.g., Lindestam et al., J. Immunol. 188(10):5020-31 (2012), which is incorporated herein in its entirety. In some aspects, the immune response is against ESAT6. In some aspects, the immune response is against TB10.4. In some aspects, the immune response is against CFP10. In some aspects, the immune response is against Rv2031 (hspX). In some aspects, the immune response is against Rv2654c (TB7.7). In some aspects, the immune response is against Rv1038c (EsxJ). In some aspects, the immune response is against an epitope selected from the group consisting of ESAT6, TB10.4 (ESAT-6-like protein EsxH; cfp7), CFP10, Rv2031 (hspX), Rv2654c (TB7.7), Rv1038c (EsxJ), and any combination thereof. See also Skjot et al., Infection and Immunity, 70 (10), 5446-5453 (October 2002), Hoang et al. PLOS One, 8 (12): 1-16, (December 2013), Billeskov et al., Eur. J. Immunol. 40: 1342-1354 (2010), and Hervas-Stubbs et al., Infection and Immunity, 74 (6): 3396-3407 (June 2006) for TB antigens, which are incorporated by reference in their entireties.

In some aspects, the immune response comprises a CD4 T cell response. In some aspects, the immune response comprises a CD8 T cell response. In some aspects, the immune response comprises a CD4 T cell response and CD8 T cell response. In certain aspects, the immune response comprises a CD4 T cell immune response with effector function that is specific to the ESAT6 antigen. In some aspects, the immune response comprises a CD8 T-cell immune response that is specific to the TB10.4 antigen.

In some aspects, the immune response comprises a T-cell immune response with effector function that is specific to a particular epitope of a TB antigen, e.g., a particular epitope of ESAT6 or TB10.4. In some aspects, the ESAT6 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in MTEQQWNFAGIEAAASAIQGNVTSIHSLLDEGKQSLTKLAAAWGGSGSEAYQGVQQKWDATATELNNALQN LARTISEAGQAMASTEGNVTGMFA (SEQ ID NO: 370). In some aspects, wherein the TB10.4 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in MSQIMYNYPAMLGHAGDMAGYAGTLQSLGAEIAVEQAALQSAWQGDTGITYQAWQAQWNQAMEDLVRA YHAMSSTHEANTMAMMARDTAEAAKWGG (GenBank: ANZ80905.1; SEQ ID NO: 371).

In some aspects, the EV, e.g., the exosome, further comprises a TB antigen. The TB antigen and IL-12 can be in the same EV or in separate EVs, an EV comprising IL-12 and an EV comprising a TB antigen. Accordingly, some aspects of the present disclosure are directed to methods of administering an EV comprising IL-12 and a TB antigen. Other aspects of the disclosure include methods of administering an EV comprising IL-12 and an EV comprising a TB antigen. The TB antigen can be within the lumen of the exosome, linked to or associated with the luminal surface of the exosome, or linked to or associated with the exterior surface of the exosome. The TB antigen can be any TB antigen known in the art, including but not limited to a TB antigen selected from the group consisting of ESAT6, TB10.4 (ESAT-6-like protein EsxH; cfp7), CFP10, Rv2031 (hspX), Rv2654c (TB7.7), Rv1038c (EsxJ), and any combination thereof. In some aspects, the EV further comprises ESAT6 or a fragment, e.g., epitope, thereof. In some aspects, the EV further comprises TB10.4 or a fragment, e.g., epitope, thereof.

In certain aspects of the disclosure, an EV disclosed herein is administered with a TB antigen disclosed herein or an EV comprising the TB antigen. In some aspects, the EV is administered prior to the TB antigen or the EV comprising the TB antigen. In some aspects, the EV is administered after the TB antigen or the EV comprising the TB antigen. In some aspects, the EV is administered concurrently with the TB antigen or the EV comprising the TB antigen. In some aspects, the subject is primed by administration of the TB antigen or an EV comprising the TB antigen.

In some aspects, the subject is further administered an additional therapeutic agent. The additional therapeutic agent can be any agent known in the art to treat TB, such as isoniazid, ifampin, pyrazinamide, ethambutol, or any combination thereof.

In some aspects, the EVs are administered intravenously to the circulatory system of the subject. In some aspects, the EVs are infused in suitable liquid and administered into a vein of the subject.

In some aspects, the EVs are administered intra-arterialy to the circulatory system of the subject. In some aspects, the EVs are infused in suitable liquid and administered into an artery of the subject.

In some aspects, the EVs are administered to the subject by intrathecal administration. In some aspects, the EVs are administered via an injection into the spinal canal, or into the subarachnoid space so that it reaches the cerebrospinal fluid (CSF).

In some aspects, the EVs are administered to the subject by intratracheal inhalation and/or intratracheal instillation.

In some aspects, the EVs are administered to the subject by intranasal administration. In some aspects, the EVs can be insufflated through the nose in a form of either topical administration or systemic administration. In certain aspects, the EVs are administered as nasal spray.

In some aspects, the EVs are administered to the subject by intraperitoneal administration. In some aspects, the EVs are infused in suitable liquid and injected into the peritoneum of the subject. In some aspects, the intraperitoneal administration results in distribution of the EVs to the lymphatics. In some aspects, the intraperitoneal administration results in distribution of the EVs to the thymus, spleen, and/or bone marrow. In some aspects, the intraperitoneal administration results in distribution of the EVs to one or more lymph nodes. In some aspects, the intraperitoneal administration results in distribution of the EVs to one or more of the cervical lymph node, the inguinal lymph node, the mediastinal lymph node, or the sternal lymph node. In some aspects, the intraperitoneal administration results in distribution of the EVs to the pancreas.

In some aspects, the EVs, e.g., exosomes, are administered to the subject by periocular administration. In some aspects, the EVs, e.g., exosomes, are injected into the periocular tissues. Periocular drug administration includes the routes of subconjunctival, anterior sub-Tenon's, posterior sub-Tenon's, and retrobulbar administration.

### III. Extracellular Vesicles, e.g., Exosomes

Disclosed herein are EVs, e.g., exosomes, capable of regulating the immune system of a subject. The EVs, e.g., exosomes, useful in the present disclosure have been engineered to produce multiple agents together (IL-12 and/or a TB antigen in a single EV, e.g., exosome, IL-12 and/or a TB in a single EV). In some aspects, an EV, e.g., exosome, comprises (i) IL-12 and (ii) a TB antigen. In other aspects, an EV comprises IL-12 and another EV comprises a TB antigen. In some aspects, an antigen is not expressed on major histocompatibility complex I and/or II molecules. In other aspects, while an antigen in the EV, e.g., exosome, is not expressed as MHC class I or II complex, the EV, e.g., exosome, can still contain MHC class I/II molecules on the surface of the EV, e.g., exosome. Accordingly, in certain aspects, EVs, e.g., exosomes, disclosed herein do not directly interact with T-cell receptors (TCRs) of T cells to induce an immune response against the antigen. Similarly, in certain aspects, EVs, e.g., exosomes, of the present disclosure do not transfer the antigen directly to the surface of the target cell (e.g., dendritic cell) through cross-dressing. Cross-dressing is a mechanism commonly used by EVs, e.g., exosomes, derived from dendritic cells (DEX) to induce T cell activation. See Pitt, J. M., et al., J Clin Invest 126(4): 1224-32 (2016). In other aspects, the EVs, e.g., exosomes, of the present disclosure are engulfed by antigen presenting cells and can be expressed on the surface of the antigen presenting cells as MHC class I and/or MHC class II complex.

As described supra, EVs, e.g., exosomes, described herein are extracellular vesicles with a diameter between about 20-300 nm. In certain aspects, an EV, e.g., exosome, of the present disclosure has a diameter between about 20-290 nm, 20-280 nm, 20-270 nm, 20-260 nm, 20-250 nm, 20-240 nm, 20-230 nm, 20-220 nm, 20-210 nm, 20-200 nm, 20-190 nm, 20-180 nm, 20-170 nm, 20-160 nm, 20-150 nm, 20-140 nm, 20-130 nm, 20-120 nm, 20-110 nm, 20-100 nm, 20-90 nm, 20-80 nm, 20-70 nm, 20-60 nm, 20-50 nm, 20-40 nm, 20-30 nm, 30-300 nm, 30-290 nm, 30-280 nm, 30-270 nm, 30-260 nm, 30-250 nm, 30-240 nm, 30-230 nm, 30-220 nm, 30-210 nm, 30-200 nm, 30-190 nm, 30-180 nm, 30-170 nm, 30-160 nm, 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-300 nm, 40-290 nm, 40-280 nm, 40-270 nm, 40-260 nm, 40-250 nm, 40-240 nm, 40-230 nm, 40-220 nm, 40-210 nm, 40-200 nm, 40-190 nm, 40-180 nm, 40-170 nm, 40-160 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-300 nm, 50-290 nm, 50-280 nm, 50-270 nm, 50-260 nm, 50-250 nm, 50-240 nm, 50-230 nm, 50-220 nm, 50-210 nm, 50-200 nm, 50-190 nm, 50-180 nm, 50-170 nm, 50-160 nm, 50-150 nm, 50-140 nm, 50-130 nm, 50-120 nm, 50-110 nm, 50-100 nm, 50-90 nm, 50-80 nm, 50-70 nm, 50-60 nm, 60-300 nm, 60-290 nm, 60-280 nm, 60-270 nm, 60-260 nm, 60-250 nm, 60-240 nm, 60-230 nm, 60-220 nm, 60-210 nm, 60-200 nm, 60-190 nm, 60-180 nm, 60-170 nm, 60-160 nm, 60-150 nm, 60-140 nm, 60-130 nm, 60-120 nm, 60-110 nm, 60-100 nm, 60-90 nm, 60-80 nm, 60-70 nm, 70-300 nm, 70-290 nm, 70-280 nm, 70-270 nm, 70-260 nm, 70-250 nm, 70-240 nm, 70-230 nm, 70-220 nm, 70-210 nm, 70-200 nm, 70-190 nm, 70-180 nm, 70-170 nm, 70-160 nm, 70-150 nm, 70-140 nm, 70-130 nm, 70-120 nm, 70-110 nm, 70-100 nm, 70-90 nm, 70-80 nm, 80-300 nm, 80-290 nm, 80-280 nm, 80-270 nm, 80-260 nm, 80-250 nm, 80-240 nm, 80-230 nm, 80-220 nm, 80-210 nm, 80-200 nm, 80-190 nm, 80-180 nm, 80-170 nm, 80-160 nm, 80-150 nm, 80-140 nm, 80-130 nm, 80-120 nm, 80-110 nm, 80-100 nm, 80-90 nm, 90-300 nm, 90-290 nm, 90-280 nm, 90-270 nm, 90-260 nm, 90-250 nm, 90-240 nm, 90-230 nm, 90-220 nm, 90-210 nm, 90-200 nm, 90-190 nm, 90-180 nm, 90-170 nm, 90-160 nm, 90-150 nm, 90-140 nm, 90-130 nm, 90-120 nm, 90-110 nm, 90-100 nm, 100-300 nm, 110-290 nm, 120-280 nm, 130-270 nm, 140-260 nm, 150-250 nm, 160-240 nm, 170-230 nm, 180-220 nm, or 190-210 nm. The size of the EV, e.g., exosome, described herein can be measured according to methods described, infra.

In some aspects, an EV, e.g., exosome, of the present disclosure comprises a bi-lipid membrane (“EV, e.g., exosome, membrane”), comprising an interior (luminal) surface and an exterior surface. In certain aspects, the interior (luminal) surface faces the inner core (i.e., lumen) of the EV, e.g., exosome. In certain aspects, the exterior surface can be in contact with the endosome, the multivesicular bodies, or the membrane/cytoplasm of a producer cell or a target cell

In some aspects, the EV, e.g., exosome, membrane comprises lipids and fatty acids. In some aspects, the EV, e.g., exosome, membrane comprises phospholipids, glycolipids, fatty acids, sphingolipids, phosphoglycerides, sterols, cholesterols, and phosphatidylserines.

In some aspects, the EV, e.g., exosome, membrane comprises an inner leaflet and an outer leaflet. The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine.

In some aspects, the EV, e.g., exosome, membrane comprises one or more polysaccharide, such as glycan.

In some aspects, the EV, e.g., exosome, of the present disclosure comprises IL-12, wherein IL-12 is linked to the EV via a scaffold moiety, either on the exterior surface of the EV or on the luminal surface of the EV.

In some aspects, the EV, e.g., exosome, of the present disclosure comprises both IL-12 and a TB antigen in the lumen of the EV. In other aspects, the EV comprises IL-12 on the exterior surface of the EV, optionally linked via a first scaffold moiety (e.g., Scaffold X), and a TB antigen on the exterior surface of the EV, optionally linked via a second scaffold moiety (e.g., Scaffold X), wherein the first scaffold moiety and the second scaffold moiety are the same or different. In other aspects, the EV comprises IL-12 on the exterior surface of the EV, optionally linked via a scaffold moiety (e.g., Scaffold X), and a TB antigen on the luminal surface of the EV, optionally linked via the scaffold moiety (e.g., Scaffold X). In other aspects, the EV comprises a TB antigen on the exterior surface of the EV, optionally linked via a scaffold moiety (e.g., Scaffold X), and IL-12 on the luminal surface of the EV, optionally linked via the scaffold moiety (e.g., Scaffold X). In other aspects, the EV comprises IL-12 on the exterior surface of the EV, optionally linked via a first scaffold moiety (e.g., Scaffold X), and a TB antigen on the luminal surface of the EV (e.g., Scaffold X or Scaffold Y), optionally linked via a second scaffold moiety, wherein the first scaffold moiety and the second scaffold moiety are the same or different. In other aspects, the EV comprises a TB antigen on the exterior surface of the EV, optionally linked via a first scaffold moiety (e.g., Scaffold X), and IL-12 on the luminal surface of the EV, optionally linked via a second scaffold moiety (e.g., Scaffold X or Scaffold Y), wherein the first scaffold moiety and the second scaffold moiety are the same or different. In some aspects, the EV of the present disclosure comprises IL-12 on the luminal surface of the EV (e.g., Scaffold X or Scaffold Y), optionally linked via a first scaffold moiety, and a TB antigen on the luminal surface of the EV (e.g., Scaffold X or Scaffold Y), optionally linked via a second scaffold moiety, wherein the first scaffold moiety and the second scaffold moiety are the same or different.

### III.A. Scaffold Moieties

One or more scaffold moieties can be used to anchor IL-12 and/or a TB antigen to the EV of the present disclosure. In some aspects, the IL-12 is linked to the scaffold moiety. In some aspects, the TB antigen is linked to the scaffold moiety. In some aspects, the EV comprises more than one scaffold moiety. In some aspects, the IL-12 is linked to a first scaffold moiety and the TB antigen is linked to a second scaffold moiety. In some aspects, the first scaffold moiety and the second scaffold moiety are the same type of scaffold moiety, e.g., the first and second scaffold moieties are both a Scaffold X protein. In some aspects, the first scaffold moiety and the second scaffold moiety are different types of scaffold moiety, e.g., the first scaffold moiety is a Scaffold Y protein and the second scaffold moiety is a Scaffold X protein. In some aspects, the first scaffold moiety is a Scaffold Y, disclosed herein. In some aspects, the first scaffold moiety is a Scaffold X, disclosed herein. In some aspects, the second scaffold moiety is a Scaffold Y, disclosed herein. In some aspects, the second scaffold moiety is a Scaffold X, disclosed herein.

In some aspects, the EV comprises one or more scaffold moieties, which are capable of anchoring, e.g., IL-12 and/or a TB antigen, to the EV, e.g., exosome, (e.g., either on the luminal surface or on the exterior surface). In certain aspects, the scaffold moiety is a polypeptide (“scaffold protein”). In certain aspects, the scaffold protein comprises an exosome protein or a fragment thereof. In other aspects, scaffold moieties are non-polypeptide moieties. In some aspects, scaffold proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., scaffold protein) comprises Scaffold X. In other aspects, a scaffold moiety (e.g., exosome protein) comprises Scaffold Y. In further aspects, a scaffold moiety (e.g., exosome protein) comprises both a Scaffold X and a Scaffold Y.

In some aspects, the IL-12 is linked to a scaffold moiety (e.g., Scaffold X) on the exterior surface of the EV, and the TB antigen is linked to the scaffold moiety on the luminal surface of the EV. In some aspects, the IL-12 is linked to a scaffold moiety (e.g., Scaffold X) on the luminal surface of the EV, and the TB antigen is linked to the scaffold moiety on the exterior surface of the EV. In some aspects, the TB antigen is linked to a scaffold moiety (e.g., Scaffold X) on the exterior surface of the EV, and the IL-12 is linked to a second scaffold moiety (e.g., Scaffold X) on the exterior surface of the EV. In some aspects, the TB antigen is linked to a scaffold moiety (e.g., Scaffold X) on the exterior surface of the EV, and the IL-12 is linked to a second scaffold moiety (e.g., Scaffold Y) on the luminal surface of the EV. In some aspects, the TB antigen is linked to a scaffold moiety (e.g., Scaffold X) on the exterior surface of the EV, and the IL-12 is linked to a second scaffold moiety (e.g., Scaffold X) on the luminal surface of the EV. In some aspects, the IL-12 is linked to a scaffold moiety (e.g., Scaffold X) on the exterior surface of the EV, and the TB antigen is linked to a second scaffold moiety (e.g., Scaffold X) on the luminal surface of the EV. In some aspects, the IL-12 is linked to a scaffold moiety (e.g., Scaffold X) on the exterior surface of the EV, and the TB antigen is linked to a second scaffold moiety (e.g., Scaffold Y) on the luminal surface of the EV. In some aspects, the IL-12 is linked to a scaffold moiety (e.g., Scaffold Y) on the luminal surface of the EV, and the TB antigen is linked to a second scaffold moiety (e.g., Scaffold Y) on the luminal surface of the EV. In some aspects, the IL-12 is linked to a scaffold moiety (e.g., Scaffold X) on the luminal surface of the EV, and the TB antigen is linked to a second scaffold moiety (e.g., Scaffold Y) on the luminal surface of the EV. In some aspects, the IL-12 is linked to a scaffold moiety (e.g., Scaffold Y) on the luminal surface of the EV, and the TB antigen is linked to a second scaffold moiety (e.g., Scaffold X) on the luminal surface of the EV.

### III.A.1. Scaffold X-Engineered EVs, e.g., Exosomes

In some aspects, EVs, e.g., exosomes, of the present disclosure comprise a membrane modified in its composition. For example, their membrane compositions can be modified by changing the protein, lipid, or glycan content of the membrane.

In some aspects, the surface-engineered EVs, e.g., exosomes, are generated by chemical and/or physical methods, such as PEG-induced fusion and/or ultrasonic fusion. In other aspects, the surface-engineered EVs, e.g., exosomes, are generated by genetic engineering. EVs, e.g., exosomes, produced from a genetically-modified producer cell or a progeny of the genetically-modified cell can contain modified membrane compositions. In some aspects, surface-engineered EVs, e.g., exosomes, have scaffold moiety (e.g., exosome protein, e.g., Scaffold X) at a higher or lower density (e.g., higher number) or include a variant or a fragment of the scaffold moiety.

For example, surface (e.g., Scaffold X)-engineered EVs, can be produced from a cell (e.g., HEK293 cells) transformed with an exogenous sequence encoding a scaffold moiety (e.g., exosome proteins, e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, scaffold moiety modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Scaffold moieties can be engineered to be expressed as a fusion molecule, e.g., fusion molecule of Scaffold X to an IL-12 and/or a TB antigen. For example, the fusion molecule can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to IL-12 and/or a TB antigen.

In some aspects, the surface (e.g., Scaffold X)-engineered EVs described herein demonstrate superior characteristics compared to EVs known in the art. For example, surface (e.g., Scaffold X)-engineered contain modified proteins more highly enriched on their surface than naturally occurring EVs or the EVs produced using conventional exosome proteins. Moreover, the surface (e.g., Scaffold X)-engineered EVs of the present disclosure can have greater, more specific, or more controlled biological activity compared to naturally occurring EVs or the EVs produced using conventional exosome proteins.

In some aspects, the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at Table 1 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33. In other aspects, the Scaffold X comprises the amino acid sequence of SEQ ID NO: 33, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 33 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 33.

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, 3, 4, 5, 6, or 7. In other aspects, the Scaffold X comprises the amino acid sequence of SEQ ID NO: 2, 3, 4, 5, 6, or 7, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 2, 3, 4, 5, 6, or 7 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 2, 3, 4, 5, 6, or 7.

TABLE 1
Exemplary Scaffold X Protein Sequences

| Protein | Sequence |
|---|---|
| The | MGRLASRPLLLALLSLALCRGRVVRVPTATLVRVVGTELVIPCNVSDYDGPSEQNFDWSF |
| PTGFRN | SSLGSSFVELASTWEVGFPAQLYQERLQRGEILLRRTANDAVELHIKNVQPSDQGHYKCS |
| Protein | TPSTDATVQGNYEDTVQVKVLADSLHVGPSARPPPSLSLREGEPFELRCTAASASPLHTH |
| (SEQ ID | LALLWEVHRGPARRSVLALTHEGRFHPGLGYEQRYHSGDVRLDTVGSDAYRLSVSRALSA |
| NO: 1) | DQGSYRCIVSEWIAEQGNWQEIQEKAVEVATVVIQPSVLRAAVPKNVSVAEGKELDLTCN |
| | ITTDRADDVRPEVTWSFSRMPDSTLPGSRVLARLDRDSLVHSSPHVALSHVDARSYHLLV |
| | RDVSKENSGYYYCHVSLWAPGHNRSWHKVAEAVSSPAGVGVTWLEPDYQVYLNASKVPGF |
| | ADDPTELACRVVDTKSGEANVRFTVSWYYRMNRRSDNVVTSELLAVMDGDWTLKYGERSK |
| | QRAQDGDFIFSKEHTDTFNFRIQRTTEEDRGNYYCVVSAWTKQRNNSWVKSKDVFSKPVN |
| | IFWALEDSVLVVKARQPKPFFAAGNTFEMTCKVSSKNIKSPRYSVLIMAEKPVGDLSSPN |
| | ETKYIISLDQDSVVKLENWTDASRVDGVVLEKVQEDEFRYRMYQTQVSDAGLYRCMVTAW |
| | SPVRGSLWREAATSLSNPIEIDFQTSGPIFNASVHSDTPSVIRGDLIKLFCIITVEGAAL |
| | DPDDMAFDVSWFAVHSFGLDKAPVLLSSLDRKGIVTTSRRDWKSDLSLERVSVLEFLLQV |
| | HGSEDQDFGNYYCSVTPWVKSPTGSWQKEAEIHSKPVFITVKMDVLNAFKYPLLIGVGLS |
| | TVIGLLSCLIGYCSSHWCCKKEVQETRRERRRLMSMEMD |
| The | GPIFNASVHSDTPSVIRGDLIKLFCIITVEGAALDPDDMAFDVSWFAVHSFGLDKAPVLL |
| PTGFRN | SSLDRKGIVTTSRRDWKSDLSLERVSVLEFLLQVHGSEDQDFGNYYCSVTPWVKSPTGSW |
| protein | QKEAEIHSKPVFITVKMDVLNAFKYPLLIGVGLSTVIGLLSCLIGYCSSHWCCKKEVQET |
| Fragment | RRERRRLMSMEM |
| (SEQ ID | 687-878 of SEQ ID NO: 1 |
| NO: 33) | |
| The BSG | MAAALFVLLG FALLGTHGAS GAAGFVQAPL SQQRWVGGSV ELHCEAVGSP |
| protein | VPEIQWWFEG QGPNDTCSQL WDGARLDRVH IHATYHQHAA STISIDTLVE |
| (SEQ ID | EDTGTYECRA SNDPDRNHLT RAPRVKWVRA QAVVLVLEPG TVFTTVEDLG |
| NO: 9) | SKILLTCSLN DSATEVTGHR WLKGGVVLKE DALPGQKTEF KVDSDDQWGE |
| | YSCVFLPEPM GTANIQLHGP PRVKAVKSSE HINEGETAML VCKSESVPPV |
| | TDWAWYKITD SEDKALMNGS ESRFFVSSSQ GRSELHIENL NMEADPGQYR |
| | CNGTSSKGSD QAIITLRVRS HLAALWPFLG IVAEVLVLVT IIFIYEKRRK |
| | PEDVLDDDDA GSAPLKSSGQ HQNDKGKNVR QRNSS |
| The IGSF8 | MGALRPTLLP PSLPLLLLLM LGMGCWAREV LVPEGPLYRV AGTAVSISCN |
| protein | VTGYEGPAQQ NFEWFLYRPE APDTALGIVS TKDTQFSYAV FKSRVVAGEV |
| (SEQ ID | QVQRLQGDAV VLKIARLQAQ DAGIYECHTP STDTRYLGSY SGKVELRVLP |
| NO: 14) | DVLQVSAAPP GPRGRQAPTS PPRMTVHEGQ ELALGCLART STQKHTHLAV |
| | SFGRSVPEAP VGRSTLQEVV GIRSDLAVEA GAPYAERLAA GELRLGKEGT |
| | DRYRMVVGGA QAGDAGTYHC TAAEWIQDPD GSWAQIAEKR AVLAHVDVQT |
| | LSSQLAVTVG PGERRIGPGE PLELLCNVSG ALPPAGRHAA YSVGWEMAPA |
| | GAPGPGRLVA QLDTEGVGSL GPGYEGRHIA MEKVASRTYR LRLEAARPGD |
| | AGTYRCLAKA YVRGSGTRLR EAASARSRPL PVHVREEGVV LEAVAWLAGG |
| | TVYRGETASL LCNISVRGGP PGLRLAASWW VERPEDGELS SVPAQLVGGV |
| | GQDGVAELGV RPGGGPVSVE LVGPRSHRLR LHSLGPEDEG VYHCAPSAWV |
| | QHADYSWYQA GSARSGPVTV YPYMHALDTL FVPLLVGTGV ALVTGATVLG |
| | TITCCFMKRL RKR |
| The ITGB1 | MNLQPIFWIG LISSVCCVFA QTDENRCLKA NAKSCGECIQ AGPNCGWCTN |
| protein | STFLQEGMPT SARCDDLEAL KKKGCPPDDI ENPRGSKDIK KNKNVTNRSK |
| (SEQ ID | GTAEKLKPED ITQIQPQQLV LRLRSGEPQT FTLKFKRAED YPIDLYYLMD |
| NO: 21) | LSYSMKDDLE NVKSLGTDLM NEMRRITSDF RIGFGSFVEK TVMPYISTTP |
| | AKLRNPCTSE QNCTSPFSYK NVLSLTNKGE VFNELVGKQR ISGNLDSPEG |
| | GFDAIMQVAV CGSLIGWRNV TRLLVFSTDA GFHFAGDGKL GGIVLPNDGQ |
| | CHLENNMYTM SHYYDYPSIA HLVQKLSENN IQTIFAVTEE FQPVYKELKN |
| | LIPKSAVGTL SANSSNVIQL IIDAYNSLSS EVILENGKLS EGVTISYKSY |
| | CKNGVNGTGE NGRKCSNISI GDEVQFEISI TSNKCPKKDS DSFKIRPLGF |
| | TEEVEVILQY ICECECQSEG IPESPKCHEG NGTFECGACR CNEGRVGRHC |
| | ECSTDEVNSE DMDAYCRKEN SSEICSNNGE CVCGQCVCRK RDNTNEIYSG |
| | ASNGQICNGR GICECGVCKC TDPKFQGQTC EMCQTCLGVC AEHKECVQCR |
| | AFNKGEKKDT CTQECSYFNI TKVESRDKLP QPVQPDPVSH CKEKDVDDCW |
| | FYFTYSVNGN NEVMVHVVEN PECPTGPDII PIVAGVVAGI VLIGLALLLI |
| | WKLLMIIHDR REFAKFEKEK MNAKWDTGEN PIYKSAVTTV VNPKYEGK |
| The ITGA4 | MAWEARREPG PRRAAVRETV MLLLCLGVPT GRPYNVDTES ALLYQGPHNT |
| protein | LFGYSVVLHS HGANRWLLVG APTANWLANA SVINPGAIYR CRIGKNPGQT |
| (SEQ ID | CEQLQLGSPN GEPCGKTCLE ERDNQWLGVT LSRQPGENGS IVTCGHRWKN |
| NO: 22) | IFYIKNENKL PTGGCYGVPP DLRTELSKRI APCYQDYVKK FGENFASCQA |
| | GISSFYTKDL IVMGAPGSSY WTGSLFVYNI TTNKYKAFLD KQNQVKFGSY |
| | LGYSVGAGHF RSQHTTEVVG GAPQHEQIGK AYIFSIDEKE LNILHEMKGK |
| | KLGSYFGASV CAVDLNADGF SDLLVGAPMQ STIREEGRVF VYINSGSGAV |
| | MNAMETNLVG SDKYAARFGE SIVNLGDIDN DGFEDVAIGA PQEDDLQGAI |
| | YIYNGRADGI SSTFSQRIEG LQISKSLSMF GQSISGQIDA DNNGYVDVAV |
| | GAFRSDSAVL LRTRPVVIVD ASLSHPESVN RTKFDCVENG WPSVCIDLTL |
| | CFSYKGKEVP GYIVLFYNMS LDVNRKAESP PRFYFSSNGT SDVITGSIQV |
| | SSREANCRTH QAFMRKDVRD ILTPIQIEAA YHLGPHVISK RSTEEFPPLQ |
| | PILQQKKEKD IMKKTINFAR FCAHENCSAD LQVSAKIGFL KPHENKTYLA |
| | VGSMKTLMLN VSLFNAGDDA YETTLHVKLP VGLYFIKILE LEEKQINCEV |
| | TDNSGVVQLD CSIGYIYVDH LSRIDISFLL DVSSLSRAEE DLSITVHATC |
| | ENEEEMDNLK HSRVTVAIPL KYEVKLTVHG FVNPTSFVYG SNDENEPETC |
| | MVEKMNLTFH VINTGNSMAP NVSVEIMVPN SFSPQTDKLF NILDVQTTTG |
| | ECHFENYQRV CALEQQKSAM QTLKGIVRFL SKTDKRLLYC IKADPHCLNF |
| | LCNFGKMESG KEASVHIQLE GRPSILEMDE TSALKFEIRA TGFPEPNPRV |
| | IELNKDENVA HVLLEGLHHQ RPKRYFTIVI ISSSLLLGLI VLLLISYVMW |
| | KAGFFKRQYK SILQEENRRD SWSYINSKSN DD |
| The | MELQPPEASI AVVSIPRQLP GSHSEAGVQG LSAGDDSELG SHCVAQTGLE |
| SLC3A2 | LLASGDPLPS ASQNAEMIET GSDCVTQAGL QLLASSDPPA LASKNAEVTG |
| Protein, | TMSQDTEVDM KEVELNELEP EKQPMNAASG AAMSLAGAEK NGLVKIKVAE |
| where | DEAEAAAAAK FTGLSKEELL KVAGSPGWVR TRWALLLLFW LGWLGMLAGA |
| the first | VVIIVRAPRC RELPAQKWWH TGALYRIGDL QAFQGHGAGN LAGLKGRLDY |
| Met is | LSSLKVKGLV LGPIHKNQKD DVAQTDLLQI DPNFGSKEDF DSLLQSAKKK |
| processed. | SIRVILDLTP NYRGENSWFS TQVDTVATKV KDALEFWLQA GVDGFQVRDI |
| (SEQ ID | ENLKDASSFL AEWQNITKGF SEDRLLIAGT NSSDLQQILS LLESNKDLLL |
| NO: 23) | TSSYLSDSGS TGEHTKSLVT QYLNATGNRW CSWSLSQARL LTSFLPAQLL |
| | RLYQLMLFTL PGTPVFSYGD EIGLDAAALP GQPMEAPVML WDESSFPDIP |
| | GAVSANMTVK GQSEDPGSLL SLFRRLSDQR SKERSLLHGD FHAFSAGPGL |
| | FSYIRHWDQN ERFLVVLNFG DVGLSAGLQA SDLPASASLP AKADLLLSTQ |
| | PGREEGSPLE LERLKLEPHE GLLLRFPYAA |

In some aspects, a Scaffold X comprises Basigin (the BSG protein), represented by SEQ ID NO: 9. The BSG protein is also known as 5F7, Collagenase stimulatory factor, Extracellular matrix metalloproteinase inducer (EMMN/PRIN), Leukocyte activation antigen M6, OK blood group antigen, Tumor cell-derived collagenase stimulatory factor (TCSF), or CD147. The Uniprot number for the human BSG protein is P35613. The signal peptide of the BSG protein is amino acid 1 to 21 of SEQ TD NO: 9. Amino acids 138-323 of SEQ ID NO: 9 is the extracellular domain, amino acids 324 to 344 is the transmembrane domain, and amino acids 345 to 385 of SEQ ID NO: 9 is the cytoplasmic domain.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 22 to 385 of SEQ ID NO: 9. In some aspects, the fragments of BSG polypeptide lack one or more functional or structural domains, such as IgV, e.g., amino acids 221 to 315 of SEQ ID NO: 9. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 10, 11, or 12. In other aspects, the Scaffold X comprises the amino acid sequence of SEQ ID NO: 10, 11, or 12, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 10, 11, or 12 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 10, 11, or 12.

In some aspects, a Scaffold X comprises Immunoglobulin superfamily member 8 (IgSF8 or the IGSF8 protein), which is also known as CD81 partner 3, Glu-Trp-Ile EWI motif-containing protein 2 (EWI-2), Keratinocytes-associated transmembrane protein 4 (KCT-4), LIR-D1, Prostaglandin regulatory-like protein (PGRL) or CD316. The full length human IGSF8 protein is accession no. Q969P0 in Uniprot and is shown as SEQ ID NO: 14 herein. The human IGSF8 protein has a signal peptide (amino acids 1 to 27 of SEQ ID NO: 14), an extracellular domain (amino acids 28 to 579 of SEQ ID NO: 14), a transmembrane domain (amino acids 580 to 600 of SEQ ID NO: 14), and a cytoplasmic domain (amino acids 601 to 613 of SEQ ID NO: 14).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of SEQ ID NO: 14. In some aspects, the IGSF8 protein lack one or more functional or structural domains, such as IgV. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 15, 16, 17, or 18. In other aspects, the Scaffold X comprises the amino acid sequence of SEQ ID NO: 15, 16, 17, or 18, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID 15, 16, 17, or 18 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 15, 16, 17, or 18.

In some aspects, a Scaffold X for the present disclosure comprises Immunoglobulin superfamily member 3 (IgSF3 or the IGSF3 protein), which is also known as Glu-Trp-Ile EWI motif-containing protein 3 (EWI-3), and is shown as the amino acid sequence of SEQ ID NO: 20. The human IGSF3 protein has a signal peptide (amino acids 1 to 19 of SEQ ID NO: 20), an extracellular domain (amino acids 20 to 1124 of SEQ ID NO: 20), a transmembrane domain (amino acids 1125 to 1145 of SEQ ID NO: 20), and a cytoplasmic domain (amino acids 1146 to 1194 of SEQ ID NO: 20).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 28 to 613 of SEQ ID NO: 20. In some aspects, the IGSF3 protein lack one or more functional or structural domains, such as IgV.

In some aspects, a Scaffold X for the present disclosure comprises Integrin beta-1 (the ITGB1 protein), which is also known as Fibronectin receptor subunit beta, Glycoprotein IIa (GPIIA), VLA-4 subunit beta, or CD29, and is shown as the amino acid sequence of SEQ ID NO: 21. The human ITGB1 protein has a signal peptide (amino acids 1 to 20 of SEQ ID NO: 21), an extracellular domain (amino acids 21 to 728 of SEQ ID NO: 21), a transmembrane domain (amino acids 729 to 751 of SEQ ID NO: 21), and a cytoplasmic domain (amino acids 752 to 798 of SEQ ID NO: 21).

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 21 to 798 of SEQ ID NO: 21. In some aspects, the ITGB1 protein lack one or more functional or structural domains, such as IgV.

In other aspects, the Scaffold X comprises the ITGA4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 22 without the signal peptide (amino acids 1 to 33 of SEQ ID NO: 22). In some aspects, the ITGA4 protein lacks one or more functional or structural domains, such as IgV.

In other aspects, the Scaffold X comprises the SLC3A2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 23 without the signal peptide. In some aspects, the SLC3A2 protein lacks one or more functional or structural domains, such as IgV.

In other aspects, the Scaffold X comprises the ATP1A1 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 24 without the signal peptide. In some aspects, the ATP1A1 protein lacks one or more functional or structural domains, such as IgV.

In other aspects, the Scaffold X comprises the ATP1A2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 25 without the signal peptide. In some aspects, the ATP1A2 protein lacks one or more functional or structural domains, such as IgV.

In other aspects, the Scaffold X comprises the ATP1A3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 26 without the signal peptide. In some aspects, the ATP1A3 protein lacks one or more functional or structural domains, such as IgV.

In other aspects, the Scaffold X comprises the ATP1A4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 27 without the signal peptide. In some aspects, the ATP1A4 protein lacks one or more functional or structural domains, such as IgV.

In other aspects, the Scaffold X comprises the ATP1A5 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 28 without the signal peptide. In some aspects, the ATP1A5 protein lacks one or more functional or structural domains, such as IgV.

In other aspects, the Scaffold X comprises the ATP2B1 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 29 without the signal peptide. In some aspects, the ATP2B1 protein lacks one or more functional or structural domains, such as IgV.

In other aspects, the Scaffold X comprises the ATP2B2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 30 without the signal peptide. In some aspects, the ATP2B2 protein lacks one or more functional or structural domains, such as IgV.

In other aspects, the Scaffold X comprises the ATP2B3 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 31 without the signal peptide. In some aspects, the ATP2B3 protein lacks one or more functional or structural domains, such as IgV.

In other aspects, the Scaffold X comprises the ATP2B4 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 32 without the signal peptide. In some aspects, the ATP2B4 protein lacks one or more functional or structural domains, such as IgV.

In other aspects, the Scaffold X comprises the IGSF2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 34 without the signal peptide. In some aspects, the IGSF2 protein lacks one or more functional or structural domains, such as IgV.

Non-limiting examples of other Scaffold X proteins can be found at U.S. Pat. No. 10,195,290B1, issued Feb. 5, 2019, which is incorporated by reference in its entireties.

In some aspects, the sequence encodes a fragment of the scaffold moiety lacking at least 5, 10, 50, 100, 200, 300, 400, 500, 600, 700, or 800 amino acids from the N-terminus of the native protein. In some aspects, the sequence encodes a fragment of the scaffold moiety lacking at least 5, 10, 50, 100, 200, 300, 400, 500, 600, 700, or 800 amino acids from the C-terminus of the native protein. In some aspects, the sequence encodes a fragment of the scaffold moiety lacking at least 5, 10, 50, 100, 200, 300, 400, 500, 600, 700, or 800 amino acids from both the N-terminus and C-terminus of the native protein. In some aspects, the sequence encodes a fragment of the scaffold moiety lacking one or more functional or structural domains of the native protein.

In some aspects, the scaffold moieties, e.g., Scaffold X, e.g., a PTGFRN protein, are linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, Scaffold X can be used to link any moiety, e.g., an IL-12 and/or a TB antigen, to the luminal surface and on the exterior surface of the EV, e.g., exosome, at the same time. For example, the PTGFRN polypeptide can be used to link an IL-12 and/or a TB antigen inside the lumen (e.g., on the luminal surface) in addition to the exterior surface of the EV, e.g., exosome. Therefore, in certain aspects, Scaffold X can be used for dual purposes, e.g., a TB antigen on the luminal surface and an IL-12 on the exterior surface of the EV, e.g., exosome; or a TB antigen on the exterior surface of the EV, e.g., exosome, and an IL-12 on the luminal surface. In some aspects, Scaffold X is a scaffold protein that is capable of anchoring the IL-12 and/or the TB antigen on the luminal surface of the EV and/or on the exterior surface of the EV.

### III.A.2. Scaffold Y-Engineered EVs, e.g., Exosomes

In some aspects, EVs, e.g., exosomes, of the present disclosure comprise an internal space (i.e., lumen) that is different from that of the naturally occurring EVs. For example, the EV can be changed such that the composition in the luminal surface of the EV, e.g., exosome has the protein, lipid, or glycan content different from that of the naturally-occurring exosomes.

In some aspects, engineered EVs, e.g., exosomes, can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the EV, e.g., exosome. Various modifications or fragments of the exosome protein that can be expressed on the luminal surface of the EV, e.g., exosome, can be used for the aspects of the present disclosure.

In some aspects, the exosome proteins that can change the luminal surface of the EVs, e.g., exosomes, include, but are not limited to, the myristoylated alanine rich Protein Kinase C substrate (MARCKS) protein, the myristoylated alanine rich Protein Kinase C substrate like 1 (MARCKSL1) protein, the brain acid soluble protein 1 (BASP1) protein, or any combination thereof.

In some aspects, Scaffold Y comprises the MARCKS protein (Uniprot accession no. P29966). The MARCKS protein is also known as protein kinase C substrate, 80 kDa protein, light chain. The full-length human MARCKS protein is 332 amino acids in length and comprises a calmodulin-binding domain at amino acid residues 152-176. In some aspects, Scaffold Y comprises the MARCKSL1 protein (Uniprot accession no. P49006). The MARCKSL1 protein is also known as MARCKS-like protein 1, and macrophage myristoylated alanine-rich C kinase substrate. The full-length human MARCKSL1 protein is 195 amino acids in length. The MARCKSL1 protein has an effector domain involved in lipid-binding and calmodulin-binding at amino acid residues 87-110. In some aspects, the Scaffold Y comprises the BASP1 protein (Uniprot accession number P80723). The BASP1 protein is also known as 22 kDa neuronal tissue-enriched acidic protein or neuronal axonal membrane protein NAP-22. The full-length human BASP1 protein sequence (isomer 1) is 227 amino acids in length. An isomer produced by an alternative splicing is missing amino acids 88 to 141 from SEQ ID NO: 49 (isomer 1). Table 2 provides the full-length sequences for the exemplary Scaffold Y disclosed herein (i.e., the MARCKS, MARCKSL1, and BASP1 proteins).

TABLE 2
Exemplary Scaffold Y Protein Sequences

| Protein | Sequence |
|---|---|
| The MARCKS | MGAQFSKTAA KGEAAAERPG EAAVASSPSK ANGQENGHVK VNGDASPAAA |
| protein | ESGAKEELQA NGSAPAADKE EPAAAGSGAA SPSAAEKGEP AAAAAPEAGA |
| (SEQ ID NO: | SPVEKEAPAE GEAAEPGSPT AAEGEAASAA SSTSSPKAED GATPSPSNET |
| 47) | PKKKKKRFSF KKSFKLSGFS FKKNKKEAGE GGEAEAPAAE GGKDEAAGGA |
| | AAAAAEAGAA SGEQAAAPGE EAAAGEEGAA GGDPQEAKPQ EAAVAPEKPP |
| | ASDETKAAEE PSKVEEKKAE EAGASAAACE APSAAGPGAP PEQRAAPAEE |
| | PAAAAASSAC AAPSQEAQPE CSPEAPPAEA AE |
| The | MGSQSSKAPR GDVTAEEAAG ASPAKANGQE NGHVKSNGDL SPKGEGESPP |
| MARCKSL1 | VNGTDEAAGA TGDAIEPAPP SQGAEAKGEV PPKETPKKKK KFSFKKPFKL |
| protein | SGLSFKRNRK EGGGDSSASS PTEEEQEQGE IGACSDEGTA QEGKAAATPE |
| (SEQ ID NO: | SQEPQAKGAE ASAASEEEAG PQATEPSTPS GPESGPTPAS AEQNE |
| 48) | |
| The BASP1 | MGGKLSKKKK GYNVNDEKAK EKDKKAEGAA TEEEGTPKES EPQAAAEPAE |
| protein | AKEGKEKPDQ DAEGKAEEKE GEKDAAAAKE EAPKAEPEKT EGAAEAKAEP |
| (SEQ ID NO: | PKAPEQEQAA PGPAAGGEAP KAAEAAAAPA ESAAPAAGEE PSKEEGEPKK |
| 49) | TEAPAAPAAQ ETKSDGAPAS DSKPGSSEAA PSSKETPAAT EAPSSTPKAQ |
| | GPAASAEEPK PVEAPAANSD QTVTVKE |

The mature BASP1 protein sequence is missing the first Met from SEQ TD NO: 49 and thus contains amino acids 2 to 227 of SEQ ID NO: 49. Similarly, the mature MARCKS and MARCKSL1 proteins also lack the first Met from SEQ ID NOs: 47 and 48, respectively. Accordingly, the mature MARCKS protein contains amino acids 2 to 332 of SEQ ID NO: 47. The mature MARCKSL1 protein contains amino acids 2 to 227 of SEQ TD NO: 48.

In other aspects, Scaffold Y useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 49. In other aspects, the Scaffold Y comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of SEQ ID NOs: 50-155 without Met at amino acid residue 1 of the SEQ ID NOs: 50-155. In other aspects, a Scaffold Y useful for the present disclosure comprises the amino acid sequence of SEQ ID NO: 49, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a Scaffold Y useful for the present disclosure comprises the amino acid sequence of any one of SEQ ID NOs: 50-155 without Met at amino acid residue 1 of the SEQ ID NOs: 50-155 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NOs: 50-155 without Met at amino acid residue 1 of the SEQ ID NOs: 50-155.

In some aspects, the protein sequence of any of SEQ ID NOs: 47-155 without Met at amino acid residue 1 of the SEQ ID NOs: 47-155 is sufficient to be a Scaffold Y for the present disclosure (e.g., scaffold moiety linked to IL12 and/or a TB antigen.

In some aspects, a Scaffold Y useful for the present disclosure comprises a peptide with the GXKLSKKK, where X is alanine or any other amino acid (SEQ ID NO: 373). In some aspects, an EV, e.g., exosome, comprises a peptide with sequence of (G)(π)(ξ)(Φ/π)(S/A/G/N)(+)(+), wherein each parenthetical position represents an amino acid, and wherein π is any amino acid selected from the group consisting of (Pro, Gly, Ala, Ser), is any amino acid selected from the group consisting of (Asn, Gln, Ser, Thr, Asp, Glu, Lys, His, Arg), (is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). In further aspects, an exosome described herein (e.g., engineered exosome) comprises a peptide with sequence of (G)(π)(X)(Φ/π)(π)(+)(+), wherein each parenthetical position represents an amino acid, and wherein π is any amino acid selected from the group consisting of (Pro, Gly, Ala, Ser), X is any amino acid, Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). See Aasland et al., FEBS Letters 513 (2002) 141-144 for amino acid nomenclature.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to any one of SEQ ID NO: 47-155 without Met at amino acid residue 1 of the SEQ ID NOs: 47-155.

Scaffold Y-engineered EVs, e.g., exosomes described herein can be produced from a cell transformed with a sequence set forth in SEQ ID NOs: 47-155 without Met at amino acid residue 1 of the SEQ ID NOs: 47-155.

In some aspects, the Scaffold Y protein useful for the present disclosure comprises an “N-terminus domain” (ND) and an “effector domain” (ED), wherein the ND and/or the ED are associated with the luminal surface of the EV, e.g., an exosome. In some aspects, the Scaffold Y protein useful for the present disclosure comprises an intracellular domain, a transmembrane domain, and an extracellular domain; wherein the intracellular domain comprises an “N-terminus domain” (ND) and an “effector domain” (ED), wherein the ND and/or the ED are associated with the luminal surface of the EV, e.g., an exosome. As used herein the term “associated with” refers to the interaction between a scaffold protein with the luminal surface of the EV, e.g., and exosome, that does not involve covalent linking to a membrane component. For example, the scaffolds useful for the present disclosure can be associated with the luminal surface of the EV, e.g., via a lipid anchor (e.g., myristic acid), and/or a polybasic domain that interacts electrostatically with the negatively charged head of membrane phospholipids. In other aspects, the Scaffold Y protein comprises an N-terminus domain (ND) and an effector domain (ED), wherein the ND is associated with the luminal surface of the EV and the ED are associated with the luminal surface of the EV by an ionic interaction, wherein the ED comprises at least two, at least three, at least four, at least five, at least six, or at least seven contiguous basic amino acids, e.g., lysines (Lys), in sequence.

In other aspects, the Scaffold Y protein comprises an N-terminus domain (ND) and an effector domain (ED), wherein the ND is associated with the luminal surface of the EV, e.g., exosome, and the ED is associated with the luminal surface of the EV by an ionic interaction, wherein the ED comprises at least two, at least three, at least four, at least five, at least six, or at least seven contiguous basic amino acids, e.g., lysines (Lys), in sequence.

In some aspects, the ND is associated with the luminal surface of the EV, e.g., an exosome, via lipidation, e.g., via myristoylation. In some aspects, the ND has Gly at the N terminus. In some aspects, the N-terminal Gly is myristoylated.

In some aspects, the ED is associated with the luminal surface of the EV, e.g., an exosome, by an ionic interaction. In some aspects, the ED is associated with the luminal surface of the EV, e.g., an exosome, by an electrostatic interaction, in particular, an attractive electrostatic interaction.

In some aspects, the ED comprises (i) a basic amino acid (e.g., lysine), or (ii) two or more basic amino acids (e.g., lysine) next to each other in a polypeptide sequence. In some aspects, the basic amino acid is lysine (Lys; K), arginine (Arg, R), or Histidine (His, H). In some aspects, the basic amino acid is (Lys)n, wherein n is an integer between 1 and 10.

In other aspects, the ED comprises at least a lysine and the ND comprises a lysine at the C terminus if the N terminus of the ED is directly linked to lysine at the C terminus of the ND, i.e., the lysine is in the N terminus of the ED and is fused to the lysine in the C terminus of the ND. In other aspects, the ED comprises at least two lysines, at least three lysines, at least four lysines, at least five lysines, at least six lysines, or at least seven lysines when the N terminus of the ED is linked to the C terminus of the ND by a linker, e.g., one or more amino acids.

In some aspects, the ED comprises K, KK, KKK, KKKK (SEQ ID NO: 205), KKKKK (SEQ ID NO: 206), R, RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof. In some aspects, the ED comprises KK, KKK, KKKK (SEQ ID NO: 205), KKKKK (SEQ ID NO: 206), or any combination thereof. In some aspects, the ND comprises the amino acid sequence as set forth in G:X2:X3:X4:X5:X6, wherein G represents Gly; wherein “:” represents a peptide bond; wherein each of the X2 to the X6 independently represents an amino acid; and wherein the X6 represents a basic amino acid. In some aspects, the X6 amino acid is selected is selected from the group consisting of Lys, Arg, and His. In some aspects, the X5 amino acid is selected from the group consisting of Pro, Gly, Ala, and Ser. In some aspects, the X2 amino acid is selected from the group consisting of Pro, Gly, Ala, and Ser. In some aspects, the X4 is selected from the group consisting of Pro, Gly, Ala, Ser, Val, Ile, Leu, Phe, Trp, Tyr, Gln, and Met.

In some aspects, the Scaffold Y protein comprises an N-terminus domain (ND) and an effector domain (ED), wherein the ND comprises the amino acid sequence as set forth in G:X2:X3:X4:X5:X6, wherein G represents Gly; wherein “:” represents a peptide bond; wherein each of the X2 to the X6 is independently an amino acid; wherein the X6 comprises a basic amino acid, and wherein the ED is linked to X6 by a peptide bond and comprises at least one lysine at the N terminus of the ED.

In some aspects, the ND of the Scaffold Y protein comprises the amino acid sequence of G:X2:X3:X4:X5:X6, wherein G represents Gly; “:” represents a peptide bond; the X2 represents an amino acid selected from the group consisting of Pro, Gly, Ala, and Ser; the X3 represents any amino acid; the X4 represents an amino acid selected from the group consisting of Pro, Gly, Ala, Ser, Val, Ile, Leu, Phe, Trp, Tyr, Gln, and Met; the X5 represents an amino acid selected from the group consisting of Pro, Gly, Ala, and Ser; and the X6 represents an amino acid selected from the group consisting of Lys, Arg, and His.

In some aspects, the X3 amino acid is selected from the group consisting of Asn, Gln, Ser, Thr, Asp, Glu, Lys, His, and Arg.

In some aspects, the ND and ED are joined by a linker. In some aspects, the linker comprises one or more amino acids. In some aspects, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. Generally, linkers provide flexibility or prevent/ameliorate steric hindrances. Linkers are not typically cleaved; however, in certain aspects, such cleavage can be desirable. Accordingly, in some aspects a linker can comprise one or more protease-cleavable sites, which can be located within the sequence of the linker or flanking the linker at either end of the linker sequence. When the ND and ED are joined by a linker, the ED comprise at least two lysines, at least three lysines, at least four lysines, at least five lysines, at least six lysines, or at least seven lysines.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the linker is a glycine/serine linker. In some aspects, the peptide linker is glycine/serine linker according to the formula [(Gly)n-Ser]m where n is any integer from 1 to 100 and m is any integer from 1 to 100. In other aspects, the glycine/serine linker is according to the formula [(Gly)x-Sery]z wherein x in an integer from 1 to 4, y is 0 or 1, and z is an integer from 1 to 50. In some aspects, the peptide linker comprises the sequence Gn, where n can be an integer from 1 to 100. In some aspects, the peptide linker can comprise the sequence (GlyAla)n, wherein n is an integer between 1 and 100. In other aspects, the peptide linker can comprise the sequence (GlyGlySer)n, wherein n is an integer between 1 and 100.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

In other aspects, the peptide linker can comprise non-naturally occurring amino acids. In yet other aspects, the peptide linker can comprise naturally occurring amino acids occurring in a linear sequence that does not occur in nature. In still other aspects, the peptide linker can comprise a naturally occurring polypeptide sequence.

The present disclosure also provides an isolated extracellular vesicle (EV), e.g., an exosome, comprising IL-12 and/or a TB antigen linked to a Scaffold Y protein, wherein the Scaffold Y protein comprises ND ED, wherein: ND comprises G:X2:X3:X4:X5:X6; wherein: G represents Gly; “:” represents a peptide bond; X2 represents an amino acid selected from the group consisting of Pro, Gly, Ala, and Ser; X3 represents any amino acid; X4 represents an amino acid selected from the group consisting of Pro, Gly, Ala, Ser, Val, Ile, Leu, Phe, Trp, Tyr, Glu, and Met; X5 represents an amino acid selected from the group consisting of Pro, Gly, Ala, and Ser; X6 represents an amino acid selected from the group consisting of Lys, Arg, and His; represents an optional linker; and ED is an effector domain comprising (i) at least two contiguous lysines (Lys), which is linked to the X6 by a peptide bond or one or more amino acids or (ii) at least one lysine, which is directly linked to the X6 by a peptide bond.

In some aspects, the X2 amino acid is selected from the group consisting of Gly and Ala. In some aspects, the X3 amino acid is Lys. In some aspects, the X4 amino acid is Leu or Glu. In some aspects, the X5 amino acid is selected from the group consisting of Ser and Ala. In some aspects, the X6 amino acid is Lys. In some aspects, the X2 amino acid is Gly, Ala, or Ser; the X3 amino acid is Lys or Glu; the X4 amino acid is Leu, Phe, Ser, or Glu; the X5 amino acid is Ser or Ala; and X6 amino acid is Lys. In some aspects, the “-” linker comprises a peptide bond or one or more amino acids.

In some aspects, the ED in the scaffold protein comprises Lys (K), KK, KKK, KKKK (SEQ ID NO: 205), KKKKK (SEQ ID NO: 206), Arg (R), RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof.

In some aspects, the Scaffold Y protein comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), or (v) any combination thereof.

In some aspects, the ND in the Scaffold Y protein comprises an amino acid sequence selected from the group consisting of (i) GGKLSK (SEQ ID NO: 215), (ii) GAKLSK (SEQ ID NO: 216), (iii) GGKQSK (SEQ ID NO: 217), (iv) GGKLAK (SEQ ID NO: 218), or (v) any combination thereof and the ED in the scaffold protein comprises K, KK, KKK, KKKG (SEQ ID NO: 219), KKKGY (SEQ ID NO: 220), KKKGYN (SEQ ID NO: 221), KKKGYNV (SEQ ID NO: 222), KKKGYNVN (SEQ ID NO: 223), KKKGYS (SEQ ID NO: 224), KKKGYG (SEQ ID NO: 225), KKKGYGG (SEQ ID NO: 226), KKKGS (SEQ ID NO: 227), KKKGSG (SEQ ID NO: 228), KKKGSGS (SEQ ID NO: 229), KKKS (SEQ ID NO: 230), KKKSG (SEQ ID NO: 231), KKKSGG (SEQ ID NO: 232), KKKSGGS (SEQ ID NO: 233), KKKSGGSG (SEQ ID NO: 234), KKSGGSGG (SEQ ID NO: 235), KKKSGGSGGS (SEQ ID NO: 236), KRFSFKKS (SEQ ID NO: 237).

In some aspects, the polypeptide sequence of a Scaffold Y protein useful for the present disclosure consists of an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), or (v) any combination thereof.

In some aspects, the Scaffold Y protein comprises an amino acid sequence selected from the group consisting of (i) GGKLSKKK (SEQ ID NO: 238), (ii) GGKLSKKS (SEQ ID NO: 239), (iii) GAKLSKKK (SEQ ID NO: 240), (iv) GAKLSKKS (SEQ ID NO: 241), (v) GGKQSKKK (SEQ ID NO: 242), (vi) GGKQSKKS (SEQ ID NO: 243), (vii) GGKLAKKK (SEQ ID NO: 244), (viii) GGKLAKKS (SEQ ID NO: 245), and (ix) any combination thereof.

In some aspects, the polypeptide sequence of a Scaffold Y protein useful for the present disclosure consists of an amino acid sequence selected from the group consisting of (i) GGKLSKKK (SEQ ID NO: 238), (ii) GGKLSKKS (SEQ ID NO: 239), (iii) GAKLSKKK (SEQ ID NO: 240), (iv) GAKLSKKS (SEQ ID NO: 241), (v) GGKQSKKK (SEQ ID NO: 242), (vi) GGKQSKKS (SEQ ID NO: 243), (vii) GGKLAKKK (SEQ ID NO: 244), (viii) GGKLAKKS (SEQ ID NO: 245), and (ix) any combination thereof.

In some aspects, the Scaffold Y protein is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least 31, at least about 32, at least about 33, at least about 34, at least about 35, at least about 36, at least about 37, at least about 38, at least about 39, at least about 39, at least about 40, at least about 41, at least about 42, at least about 43, at least about 44, at least about 50, at least about 46, at least about 47, at least about 48, at least about 49, at least about 50, at least about 55, at least about 60, at least about 65, at least about 70, at least about 75, at least about 80, at least 85, at least about 90, at least about 95, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, at least about 195, at least about 200, at least about 205, at least about 210, at least about 215, at least about 220, at least about 225, at least about 230, at least about 235, at least about 240, at least about 245, at least about 250, at least about 255, at least about 260, at least about 265, at least about 270, at least about 275, at least about 280, at least about 285, at least about 290, at least about 295, at least about 300, at least about 305, at least about 310, at least about 315, at least about 320, at least about 325, at least about 330, at least about 335, at least about 340, at least about 345, or at least about 350 amino acids in length.

In some aspects, the Scaffold Y protein is between about 5 and about 10, between about 10 and about 20, between about 20 and about 30, between about 30 and about 40, between about 40 and about 50, between about 50 and about 60, between about 60 and about 70, between about 70 and about 80, between about 80 and about 90, between about 90 and about 100, between about 100 and about 110, between about 110 and about 120, between about 120 and about 130, between about 130 and about 140, between about 140 and about 150, between about 150 and about 160, between about 160 and about 170, between about 170 and about 180, between about 180 and about 190, between about 190 and about 200, between about 200 and about 210, between about 210 and about 220, between about 220 and about 230, between about 230 and about 240, between about 240 and about 250, between about 250 and about 260, between about 260 and about 270, between about 270 and about 280, between about 280 and about 290, between about 290 and about 300, between about 300 and about 310, between about 310 and about 320, between about 320 and about 330, between about 330 and about 340, or between about 340 and about 350 amino acids in length.

In some aspects, the Scaffold Y protein comprises (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 291).

In some aspects, the polypeptide sequence of a Scaffold Y protein useful for the present disclosure consists of (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 291).

Non-limiting examples of the Scaffold Y protein useful for the present disclosure are listed below. In some aspects, the Scaffold Y protein comprises an amino acid sequence set forth in Table 3. In some aspects, the Scaffold Y protein consists of an amino acid sequence set forth in Table 3.

TABLE 3

| SEQ ID NO: | Scaffold Protein: GX2X3X4X5X6-ED |
|---|---|
| 257 | GGKLSKKKKGYNVNDEKAKEKDKKAEGAA |
| 258 | GGKLSKKKKGYNVNDEKAKEKDKKAEGA |
| 259 | GGKLSKKKKGYNVNDEKAKEKDKKAEG |
| 260 | GGKLSKKKKGYNVNDEKAKEKDKKAE |
| 261 | GGKLSKKKKGYNVNDEKAKEKDKKA |
| 262 | GGKLSKKKKGYNVNDEKAKEKDKK |
| 263 | GGKLSKKKKGYNVNDEKAKEKDK |
| 264 | GGKLSKKKKGYNVNDEKAKEKD |
| 265 | GGKLSKKKKGYNVNDEKAKEK |
| 266 | GGKLSKKKKGYNVNDEKAKE |
| 267 | GGKLSKKKKGYNVNDEKAK |
| 268 | GGKLSKKKKGYNVNDEKA |
| 269 | GGKLSKKKKGYNVNDEK |
| 270 | GGKLSKKKKGYNVNDE |
| 271 | GGKLSKKKKGYNVND |
| 246 | GGKLSKKKKGYNVN |
| 272 | GGKLSKKKKGYNV |
| 273 | GGKLSKKKKGYN |
| 274 | GGKLSKKKKGY |
| 275 | GGKLSKKKKG |
| 276 | GGKLSKKKK |
| 238 | GGKLSKKK |
| 211 | GGKLSKK |
| 300 | GAKKSKKRFSFKKSFKLSGFSFKKNKKEA |
| 277 | GAKKSKKRFSFKKSFKLSGFSFKKNKKE |
| 278 | GAKKSKKRFSFKKSFKLSGFSFKKNKK |
| 279 | GAKKSKKRFSFKKSFKLSGFSFKKNK |
| 280 | GAKKSKKRFSFKKSFKLSGFSFKKN |
| 281 | GAKKSKKRFSFKKSFKLSGFSFKK |
| 282 | GAKKSKKRFSFKKSFKLSGFSFK |
| 283 | GAKKSKKRFSFKKSFKLSGFSF |
| 284 | GAKKSKKRFSFKKSFKLSGFS |
| 285 | GAKKSKKRFSFKKSFKLSGF |
| 286 | GAKKSKKRFSFKKSFKLSG |
| 287 | GAKKSKKRFSFKKSFKLS |
| 288 | GAKKSKKRFSFKKSFKL |
| 289 | GAKKSKKRFSFKKSFK |
| 290 | GAKKSKKRFSFKKSF |
| 291 | GAKKSKKRFSFKKS |
| 292 | GAKKSKKRFSFKK |
| 293 | GAKKSKKRFSFK |
| 294 | GAKKSKKRFSF |
| 295 | GAKKSKKRFS |
| 296 | GAKKSKKRF |
| 297 | GAKKSKKR |
| 298 | GAKKSKK |
| 301 | GAKKAKKRFSFKKSFKLSGFSFKKNKKEA |
| 348 | GAKKAKKRFSFKKSFKLSGFSFKKNKKE |
| 349 | GAKKAKKRFSFKKSFKLSGFSFKKNKK |
| 350 | GAKKAKKRFSFKKSFKLSGFSFKKNK |
| 351 | GAKKAKKRFSFKKSFKLSGFSFKKN |
| 352 | GAKKAKKRFSFKKSFKLSGFSFKK |
| 353 | GAKKAKKRFSFKKSFKLSGFSFK |
| 354 | GAKKAKKRFSFKKSFKLSGFSF |
| 355 | GAKKAKKRFSFKKSFKLSGFS |
| 356 | GAKKAKKRFSFKKSFKLSGF |
| 357 | GAKKAKKRFSFKKSFKLSG |
| 358 | GAKKAKKRFSFKKSFKLS |
| 359 | GAKKAKKRFSFKKSFKL |
| 360 | GAKKAKKRFSFKKSFK |
| 361 | GAKKAKKRFSFKKSF |
| 362 | GAKKAKKRFSFKKS |
| 363 | GAKKAKKRFSFKK |
| 364 | GAKKAKKRFSFK |
| 365 | GAKKAKKRFSF |
| 366 | GAKKAKKRFS |
| 367 | GAKKAKKRF |
| 368 | GAKKAKKR |
| 369 | GAKKAKK |
| 302 | GAQESKKKKKKRFSFKKSFKLSGFSFKK |
| 303 | GAQESKKKKKKRFSFKKSFKLSGFSFK |
| 304 | GAQESKKKKKKRFSFKKSFKLSGFSF |
| 305 | GAQESKKKKKKRFSFKKSFKLSGFS |
| 306 | GAQESKKKKKKRFSFKKSFKLSGF |
| 307 | GAQESKKKKKKRFSFKKSFKLSG |
| 308 | GAQESKKKKKKRFSFKKSFKLS |
| 309 | GAQESKKKKKKRFSFKKSFKL |
| 310 | GAQESKKKKKKRFSFKKSFK |
| 311 | GAQESKKKKKKRFSFKKSF |
| 312 | GAQESKKKKKKRFSFKKS |
| 313 | GAQESKKKKKKRFSFKK |
| 314 | GAQESKKKKKKRFSFK |
| 315 | GAQESKKKKKKRFSF |
| 316 | GAQESKKKKKKRFS |
| 317 | GAQESKKKKKKRF |
| 318 | GAQESKKKKKKR |
| 319 | GAQESKKKKKK |
| 320 | GAQESKKKKK |
| 321 | GAQESKKKK |
| 322 | GAQESKKK |
| 323 | GAQESKK |
| 324 | GSQSSKKKKKKFSFKKPFKLSGLSFKRNRK |
| 325 | GSQSSKKKKKKFSFKKPFKLSGLSFKRNR |
| 326 | GSQSSKKKKKKFSFKKPFKLSGLSFKRN |
| 327 | GSQSSKKKKKKFSFKKPFKLSGLSFKR |
| 328 | GSQSSKKKKKKFSFKKPFKLSGLSFK |
| 329 | GSQSSKKKKKKFSFKKPFKLSGLSF |
| 330 | GSQSSKKKKKKFSFKKPFKLSGLS |
| 331 | GSQSSKKKKKKFSFKKPFKLSGL |
| 332 | GSQSSKKKKKKFSFKKPFKLSG |
| 333 | GSQSSKKKKKKFSFKKPFKLS |
| 334 | GSQSSKKKKKKFSFKKPFKL |
| 335 | GSQSSKKKKKKFSFKKPFK |
| 336 | GSQSSKKKKKKFSFKKPF |
| 337 | GSQSSKKKKKKFSFKKP |
| 338 | GSQSSKKKKKKFSFKK |
| 339 | GSQSSKKKKKKFSFK |
| 340 | GSQSSKKKKKKFSF |
| 341 | GSQSSKKKKKKFS |
| 342 | GSQSSKKKKKKF |
| 343 | GSQSSKKKKKK |
| 344 | GSQSSKKKKK |
| 345 | GSQSSKKKK |
| 346 | GSQSSKKK |
| 347 | GSQSSKK |

In some aspects, the Scaffold Y protein useful for the present disclosure does not contain an N-terminal Met. In some aspects, the Scaffold Y protein comprises a lipidated amino acid, e.g., a myristoylated amino acid, at the N-terminus of the scaffold protein, which functions as a lipid anchor. In some aspects, the amino acid residue at the N-terminus of the scaffold protein is Gly. The presence of an N-terminal Gly is an absolute requirement for N-myristoylation. In some aspects, the amino acid residue at the N-terminus of the scaffold protein is synthetic. In some aspects, the amino acid residue at the N-terminus of the scaffold protein is a glycine analog, e.g., allylglycine, butylglycine, or propargylglycine.

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table:

| Modification | Modifying Group |
|---|---|
| S-Palmitoylation | 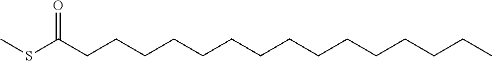 [SMILES: CCCCCCCCCCCCCCCC(=O)SC] |
| N-Palmitoylation | 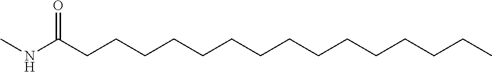 [SMILES: CCCCCCCCCCCCCCCC(=O)NC] |
| N-Myristoylation | 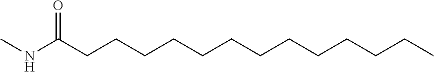 [SMILES: CCCCCCCCCCCCCC(=O)NC] |
| O-Acylation | 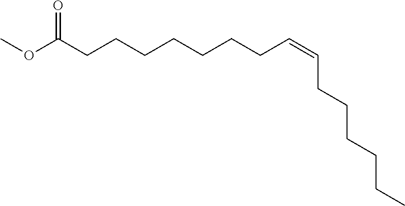 [SMILES: CCCCCC/C=C\CCCCCCCC(=O)OC] |
| Farnesylation | 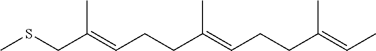 [SMILES: C/C=C(\C)CC/C=C(\C)CC/C=C(\C)CSC] |
| Geranylgeranylation | 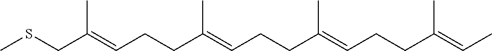 [SMILES: C/C=C(\C)CC/C=C(\C)CC/C=C(\C)CC/C=C(\C)CSC] |
| Cholesterol | 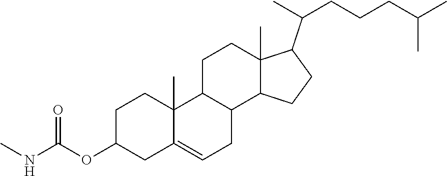 [SMILES: CNC(=O)OC1CCC2(C)C(=CCC3C2CCC2(C)C(C(C)CCCC(C)C)CCC32)C1] |

### III.C. Linkers

As described supra, extracellular vesicles (EVs) of the present disclosure (e.g., exosomes and nanovesicles) can comprises one or more linkers that link a molecule of interest (e.g., IL-12, antigen and/or a TB antigen) to the EVs (e.g., to the exterior surface or on the luminal surface). In some aspects, IL-12 and/or TB antigen is linked to the EVs directly or via a scaffold moiety (e.g., Scaffold X or Scaffold Y). In certain aspects, the IL-12 and/or the TB antigen is linked to the scaffold moiety by a linker. In certain aspects, the IL-12 and/or the TB antigen is linked to the second scaffold moiety by a linker.

In certain aspects, IL-12 and/or a TB antigen is linked to the exterior surface of an exosome via Scaffold X. In further aspects, IL-12 and/or a TB antigen is linked to the luminal surface of an exosome via Scaffold X or Scaffold Y. The linker can be any chemical moiety known in the art.

As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however, in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids. v

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

Linkers can be susceptible to cleavage (“cleavable linker”) thereby facilitating release of the biologically active molecule (e.g., IL-12 and/or a TB antigen).

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable linker.

### IV. Producer Cell for Production of Engineered Exosomes

EVs, e.g., exosomes, of the present disclosure can be produced from a cell grown in vitro or a body fluid of a subject. When exosomes are produced from in vitro cell culture, various producer cells, e.g., HEK293 cells, CHO cells, and MSCs, can be used. In certain aspects, a producer cell is not a dendritic cell, macrophage, B cell, mast cell, neutrophil, Kupffer-Browicz cell, cell derived from any of these cells, or any combination thereof.

The producer cell can be genetically modified to comprise exogenous sequences encoding IL-12 and/or a TB antigen to produce EVs described herein. The genetically-modified producer cell can contain the exogenous sequence by transient or stable transformation. The exogenous sequence can be transformed as a plasmid. In some aspects, the exogenous sequence is a vector. The exogenous sequences can be stably integrated into a genomic sequence of the producer cell, at a targeted site or in a random site. In some aspects, a stable cell line is generated for production of lumen-engineered exosomes.

The exogenous sequences can be inserted into a genomic sequence of the producer cell, located within, upstream (5′-end) or downstream (3′-end) of an endogenous sequence encoding an exosome protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous sequences can comprise a sequence encoding a scaffold moiety disclosed herein or a fragment or variant thereof. An extra copy of the sequence encoding a scaffold moiety can be introduced to produce an exosome described herein (e.g., having a higher density of a scaffold moiety on the surface or on the luminal surface of the EV, e.g., exosome). An exogenous sequence encoding a modification or a fragment of a scaffold moiety can be introduced to produce a lumen-engineered and/or surface-engineered exosome containing the modification or the fragment of the scaffold moiety.

In some aspects, a producer cell can be modified, e.g., transfected, with one or more vectors encoding a scaffold moiety linked to IL-12 and/or a TB antigen.

In some aspects, a producer cell disclosed herein is further modified to comprise an additional exogenous sequence. For example, an additional exogenous sequence can be introduced to modulate endogenous gene expression, or produce an exosome including a certain polypeptide as a payload (e.g., IL-12 and/or a TB antigen). In some aspects, the producer cell is modified to comprise two exogenous sequences, one encoding a scaffold moiety (e.g., Scaffold X and/or Scaffold Y), or a variant or a fragment thereof, and the other encoding a payload e.g., IL-12 and/or a TB antigen). In some aspects, the producer cell is modified to comprise two exogenous sequences, one encoding a scaffold moiety disclosed herein, or a variant or a fragment thereof, and the other encoding a protein conferring the additional functionalities to exosomes. In some aspects, the producer cell is further modified to comprise one, two, three, four, five, six, seven, eight, nine, or ten or more additional exogenous sequences.

In some aspects, EVs, e.g., exosomes, of the present disclosure (e.g., surface-engineered and/or lumen-engineered exosomes) can be produced from a cell transformed with a sequence encoding a full-length, mature scaffold moiety disclosed herein or a scaffold moiety linked to IL-12 and/or a TB antigen. Any of the scaffold moieties described herein can be expressed from a plasmid, an exogenous sequence inserted into the genome or other exogenous nucleic acid, such as a synthetic messenger RNA (mRNA).

### V. Pharmaceutical Compositions

Provided herein are pharmaceutical compositions comprising an EV, e.g., exosome, of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an exosome described herein. In certain aspects, the EVs, e.g., exosomes, are co-administered with one or more additional therapeutic agents, e.g., a TB antigen, in a pharmaceutically acceptable carrier. In some aspects, IL-12 and the TB antigen for the present disclosure can be administered in the same EV. In other aspects, IL-12 and the TB antigen for the present disclosure are administered in different EVs. For example, the present disclosure includes a pharmaceutical composition comprising an EV comprising IL-12 and an EV comprising a TB antigen. In some aspects, the pharmaceutical composition comprising the EV, e.g., exosome is administered prior to administration of the additional therapeutic agents. In other aspects, the pharmaceutical composition comprising the EV, e.g., exosome, is administered after the administration of the additional therapeutic agents. In further aspects, the pharmaceutical composition comprising the EV, e.g., exosome, is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs, e.g., exosomes, can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising exosomes is administered intravenously, e.g. by injection. The EVs, e.g., exosomes, can optionally be administered in combination with other therapeutic agents that are at least partly effective in treating the disease, disorder or condition for which the EVs, e.g., exosomes, are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as manitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs, e.g., exosomes, in an effective amount and in an appropriate solvent with one or more ingredients enumerated herein or known in the art, as desired. Generally, dispersions are prepared by incorporating the EVs, e.g., exosomes, into a sterile vehicle that contains a basic dispersion medium and any desired other ingredients. In the case of sterile powders for the preparation of sterile injectable solutions, methods of preparation are vacuum drying and freeze-drying that yield a powder of the active ingredient plus any additional desired ingredient from a previously sterile-filtered solution thereof. The EVs, e.g., exosomes, can be administered in the form of a depot injection or implant preparation which can be formulated in such a manner to permit a sustained or pulsatile release of the EV, e.g., exosome.

Systemic administration of compositions comprising exosomes can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising EVs, e.g., exosomes is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs, e.g., exosomes, into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs, e.g., exosomes, described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs, e.g., exosomes, described herein. In some aspects, the dosage form is formulated as a liquid suspension for intravenous injection. In some aspects, the dosage form is formulated as a liquid suspension for intratumoral injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of exosomes is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of exosomes is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or greater than 10000 mSv.

### VI. Kits

Also provided herein are kits comprising one or more exosomes described herein. In some aspects, provided herein is a pharmaceutical pack or kit comprising one or more containers filled with one or more of the ingredients of the pharmaceutical compositions described herein, such as one or more exosomes provided herein, optional an instruction for use. In some aspects, the kits contain a pharmaceutical composition described herein and any prophylactic or therapeutic agent, such as those described herein. In some aspects, the kit further comprises instructions to administer the EV according to any method disclosed herein. In some aspects, the kit is for use in the treatment of a disease or condition associated with a TB antigen. In some aspects, the disease is TB.

### VII. Methods of Producing EVs

In some aspects, the present disclosure is also directed to methods of producing EVs described herein. In some aspects, the method comprises: obtaining the EV, e.g., exosome from a producer cell, wherein the producer cell contains one or more components of the EV, e.g., exosome (e.g., IL-12 and/or TB antigen; and optionally isolating the obtained EV, e.g., exosome. In some aspects, the method comprises: modifying a producer cell by introducing one or more components of an EV disclosed herein (e.g., IL-12 and/or TB antigen); obtaining the EV, e.g., exosome from the modified producer cell; and optionally isolating the obtained EV, e.g., exosome. In further aspects, the method comprises: obtaining an EV from a producer cell; isolating the obtained EV; and modifying the isolated EV. In certain aspects, the method further comprises formulating the isolated EV into a pharmaceutical composition.

### VII.A. Methods of Modifying a Producer Cell

As described supra, in some aspects, a method of producing an EV comprises modifying a producer cell with one or more moieties (e.g., IL-12 and/or TB antigen). In certain aspects, the one or more moieties comprise IL-12 and/or a TB antigen. In some aspects, the one or more moieties further comprise a scaffold moiety disclosed herein (e.g., Scaffold X or Scaffold Y).

In some aspects, the producer cell can be a mammalian cell line, a plant cell line, an insect cell line, a fungi cell line, or a prokaryotic cell line. In certain aspects, the producer cell is a mammalian cell line. Non-limiting examples of mammalian cell lines include: a human embryonic kidney (HEK) cell line, a Chinese hamster ovary (CHO) cell line, an HT-1080 cell line, a HeLa cell line, a PERC-6 cell line, a CEVEC cell line, a fibroblast cell line, an amniocyte cell line, an epithelial cell line, a mesenchymal stem cell (MSC) cell line, and combinations thereof. In certain aspects, the mammalian cell line comprises HEK-293 cells, BJ human foreskin fibroblast cells, fHDF fibroblast cells, AGE.HN® neuronal precursor cells, CAP® amniocyte cells, adipose mesenchymal stem cells, RPTEC/TERT1 cells, or combinations thereof. In some aspects, the producer cell is a primary cell. In certain aspects, the primary cell can be a primary mammalian cell, a primary plant cell, a primary insect cell, a primary fungi cell, or a primary prokaryotic cell.

In some aspects, the producer cell is not an immune cell, such as an antigen presenting cell, a T cell, a B cell, a natural killer cell (NK cell), a macrophage, a T helper cell, or a regulatory T cell (Treg cell). In other aspects, the producer cell is not an antigen presenting cell (e.g., dendritic cells, macrophages, B cells, mast cells, neutrophils, Kupffer-Browicz cell, or a cell derived from any such cells).

In some aspects, the one or more moieties can be a transgene or mRNA, and introduced into the producer cell by transfection, viral transduction, electroporation, extrusion, sonication, cell fusion, or other methods that are known to the skilled in the art.

In some aspects, the one or more moieties is introduced to the producer cell by transfection. In some aspects, the one or more moieties can be introduced into suitable producer cells using synthetic macromolecules, such as cationic lipids and polymers (Papapetrou et al., Gene Therapy 12: S118-S130 (2005)). In some aspects, the cationic lipids form complexes with the one or more moieties through charge interactions. In some of these aspects, the positively charged complexes bind to the negatively charged cell surface and are taken up by the cell by endocytosis. In some other aspects, a cationic polymer can be used to transfect producer cells. In some of these aspects, the cationic polymer is polyethylenimine (PEI). In certain aspects, chemicals such as calcium phosphate, cyclodextrin, or polybrene, can be used to introduce the one or more moieties to the producer cells. The one or more moieties can also be introduced into a producer cell using a physical method such as particle-mediated transfection, “gene gun”, biolistics, or particle bombardment technology (Papapetrou et al., Gene Therapy 12: S118-S130 (2005)). A reporter gene such as, for example, beta-galactosidase, chloramphenicol acetyltransferase, luciferase, or green fluorescent protein can be used to assess the transfection efficiency of the producer cell.

In certain aspects, the one or more moieties are introduced to the producer cell by viral transduction. A number of viruses can be used as gene transfer vehicles, including moloney murine leukemia virus (MMLV), adenovirus, adeno-associated virus (AAV), herpes simplex virus (HSV), lentiviruses, and spumaviruses. The viral mediated gene transfer vehicles comprise vectors based on DNA viruses, such as adenovirus, adeno-associated virus and herpes virus, as well as retroviral based vectors.

In certain aspects, the one or more moieties are introduced to the producer cell by electroporation. Electroporation creates transient pores in the cell membrane, allowing for the introduction of various molecules into the cell. In some aspects, DNA and RNA as well as polypeptides and non-polypeptide therapeutic agents can be introduced into the producer cell by electroporation.

In certain aspects, the one or more moieties introduced to the producer cell by microinjection. In some aspects, a glass micropipette can be used to inject the one or more moieties into the producer cell at the microscopic level.

In certain aspects, the one or more moieties are introduced to the producer cell by extrusion.

In certain aspects, the one or more moieties are introduced to the producer cell by sonication. In some aspects, the producer cell is exposed to high intensity sound waves, causing transient disruption of the cell membrane allowing loading of the one or more moieties.

In certain aspects, the one or more moieties are introduced to the producer cell by cell fusion. In some aspects, the one or more moieties are introduced by electrical cell fusion. In other aspects, polyethylene glycol (PEG) is used to fuse the producer cells. In further aspects, sendai virus is used to fuse the producer cells.

In some aspects, the one or more moieties are introduced to the producer cell by hypotonic lysis. In such aspects, the producer cell can be exposed to low ionic strength buffer causing them to burst allowing loading of the one or more moieties. In other aspects, controlled dialysis against a hypotonic solution can be used to swell the producer cell and to create pores in the producer cell membrane. The producer cell is subsequently exposed to conditions that allow resealing of the membrane.

In some aspects, the one or more moieties are introduced to the producer cell by detergent treatment. In certain aspects, producer cell is treated with a mild detergent which transiently compromises the producer cell membrane by creating pores allowing loading of the one or more moieties. After producer cells are loaded, the detergent is washed away thereby resealing the membrane.

In some aspects, the one or more moieties introduced to the producer cell by receptor mediated endocytosis. In certain aspects, producer cells have a surface receptor which upon binding of the one or more moieties induces internalization of the receptor and the associated moieties.

In some aspects, the one or more moieties are introduced to the producer cell by filtration. In certain aspects, the producer cells and the one or more moieties can be forced through a filter of pore size smaller than the producer cell causing transient disruption of the producer cell membrane and allowing the one or more moieties to enter the producer cell.

In some aspects, the producer cell is subjected to several freeze thaw cycles, resulting in cell membrane disruption allowing loading of the one or more moieties.

### VII.B. Methods of Modifying EV, e.g., Exosome

In some aspects, a method of producing an EV, e.g., exosome, comprises modifying the isolated EV by directly introducing one or more moieties into the EVs. In certain aspects, the one or more moieties comprise IL-12 and/or a TB antigen. In some aspects, the one or more moieties comprise a scaffold moiety disclosed herein (e.g., Scaffold X or Scaffold Y).

In certain aspects, the one or more moieties are introduced to the EV by transfection. In some aspects, the one or more moieties can be introduced into the EV using synthetic macromolecules such as cationic lipids and polymers (Papapetrou et al., Gene Therapy 12: S118-5130 (2005)). In certain aspects, chemicals such as calcium phosphate, cyclodextrin, or polybrene, can be used to introduce the one or more moieties to the EV.

In certain aspects, the one or more moieties are introduced to the EV by electroporation. In some aspects, EVs are exposed to an electrical field which causes transient holes in the EV membrane, allowing loading of the one or more moieties.

In certain aspects, the one or more moieties are introduced to the EV by microinjection. In some aspects, a glass micropipette can be used to inject the one or more moieties directly into the EV at the microscopic level.

In certain aspects, the one or more moieties are introduced to the EV by extrusion.

In certain aspects, the one or more moieties are introduced to the EV by sonication. In some aspects, EVs are exposed to high intensity sound waves, causing transient disruption of the EV membrane allowing loading of the one or more moieties.

In some aspects, one or more moieties can be conjugated to the surface of the EV. Conjugation can be achieved chemically or enzymatically, by methods known in the art.

In some aspects, the EV comprises one or more moieties that are chemically conjugated. Chemical conjugation can be accomplished by covalent bonding of the one or more moieties to another molecule, with or without use of a linker. The formation of such conjugates is within the skill of artisans and various techniques are known for accomplishing the conjugation, with the choice of the particular technique being guided by the materials to be conjugated. In certain aspects, polypeptides are conjugated to the EV. In some aspects, non-polypeptides, such as lipids, carbohydrates, nucleic acids, and small molecules, are conjugated to the EV.

In some aspects, the one or more moieties are introduced to the EV by hypotonic lysis. In such aspects, the EVs can be exposed to low ionic strength buffer causing them to burst allowing loading of the one or more moieties. In other aspects, controlled dialysis against a hypotonic solution can be used to swell the EV and to create pores in the EV membrane. The EV is subsequently exposed to conditions that allow resealing of the membrane.

In some aspects, the one or more moieties are introduced to the EV by detergent treatment. In certain aspects, extracellular vesicles are treated with a mild detergent which transiently compromises the EV membrane by creating pores allowing loading of the one or more moieties. After EVs are loaded, the detergent is washed away thereby resealing the membrane.

In some aspects, the one or more moieties are introduced to the EV by receptor mediated endocytosis. In certain aspects, EVs have a surface receptor which upon binding of the one or more moieties induces internalization of the receptor and the associated moieties.

In some aspects, the one or more moieties are introduced to the EV by mechanical firing. In certain aspects, extracellular vesicles can be bombarded with one or more moieties attached to a heavy or charged particle such as gold microcarriers. In some of these aspects, the particle can be mechanically or electrically accelerated such that it traverses the EV membrane.

In some aspects, extracellular vesicles are subjected to several freeze thaw cycles, resulting in EV membrane disruption allowing loading of the one or more moieties.

### VII.C. Methods of Isolating EV, e.g., Exosome

In some aspects, methods of producing EVs disclosed herein comprises isolating the EV from the producer cells. In certain aspects, the EVs released by the producer cell into the cell culture medium. It is contemplated that all known manners of isolation of EVs are deemed suitable for use herein. For example, physical properties of EVs can be employed to separate them from a medium or other source material, including separation on the basis of electrical charge (e.g., electrophoretic separation), size (e.g., filtration, molecular sieving, etc.), density (e.g., regular or gradient centrifugation), Svedberg constant (e.g., sedimentation with or without external force, etc.). Alternatively, or additionally, isolation can be based on one or more biological properties, and include methods that can employ surface markers (e.g., for precipitation, reversible binding to solid phase, FACS separation, specific ligand binding, non-specific ligand binding, affinity purification etc.).

Isolation and enrichment can be done in a general and non-selective manner, typically including serial centrifugation. Alternatively, isolation and enrichment can be done in a more specific and selective manner, such as using EV or producer cell-specific surface markers. For example, specific surface markers can be used in immunoprecipitation, FACS sorting, affinity purification, and magnetic separation with bead-bound ligands.

In some aspects, size exclusion chromatography can be utilized to isolate the EVs. Size exclusion chromatography techniques are known in the art. Exemplary, non-limiting techniques are provided herein. In some aspects, a void volume fraction is isolated and comprises the EVs of interest. Further, in some aspects, the EVs can be further isolated after chromatographic separation by centrifugation techniques (of one or more chromatography fractions), as is generally known in the art. In some aspects, for example, density gradient centrifugation can be utilized to further isolate the extracellular vesicles. In certain aspects, it can be desirable to further separate the producer cell-derived EVs from EVs of other origin. For example, the producer cell-derived EVs can be separated from non-producer cell-derived EVs by immunosorbent capture using an antigen antibody specific for the producer cell.

In some aspects, the isolation of EVs can involve combinations of methods that include, but are not limited to, differential centrifugation, size-based membrane filtration, immunoprecipitation, FACS sorting, and magnetic separation.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1

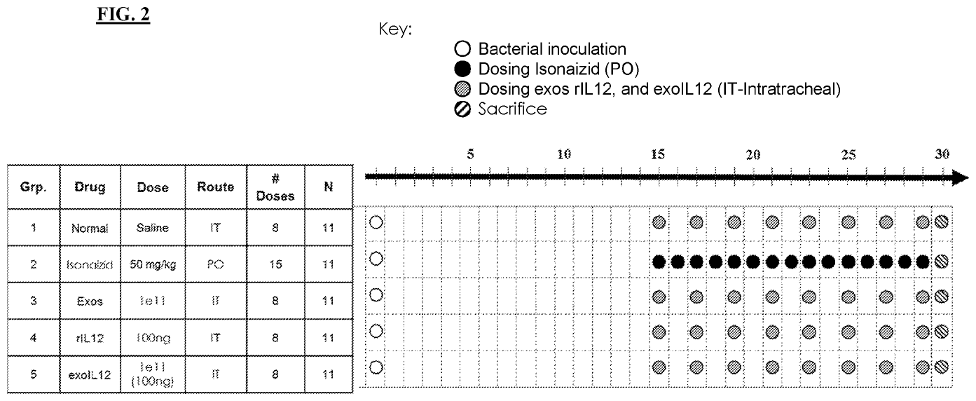

An in vivo study was conducted to determine if an exosome comprising IL-12 (“exoIL-12”) can have an impact in a tuberculosis mouse model (FIG. 2). Endpoints of this study include an analysis of viable Mycobacterium tuberculosis cells, ELISA for IFNγ levels, antigen specific T cell assay (including tetramer analysis and flow cytometry), and intracellular cytokine staining (ICCS) assay. To test the ability of loaded surface engineered exosomes to localize to cells commonly infected by Mycobacterium tuberculosis, exosomes comprising PTGFRN fused to GFP were administered to mice by inhalation. Positive GFP signal was observed in alveolar macrophages, pneumocytes, and endothelial cells of mouse lungs following administration, indicated efficient uptake of the administered exosomes.

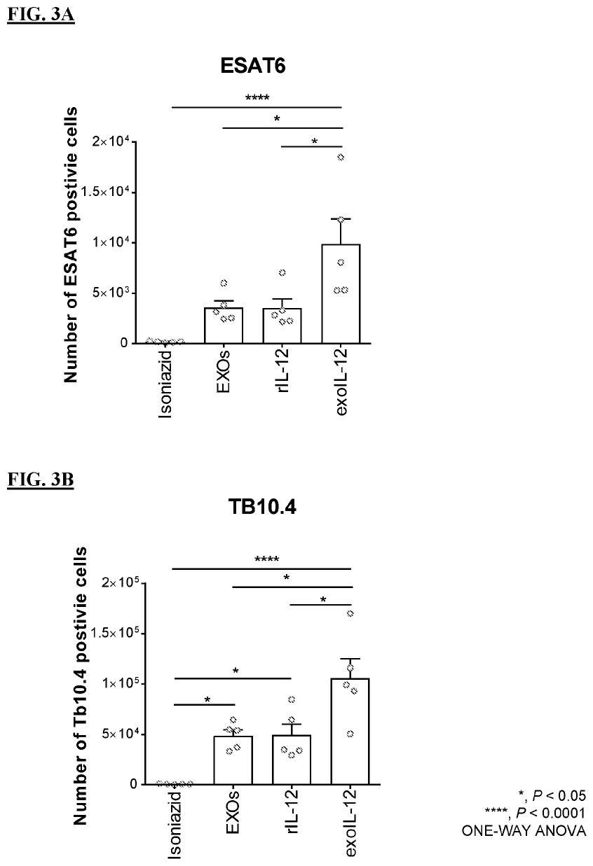

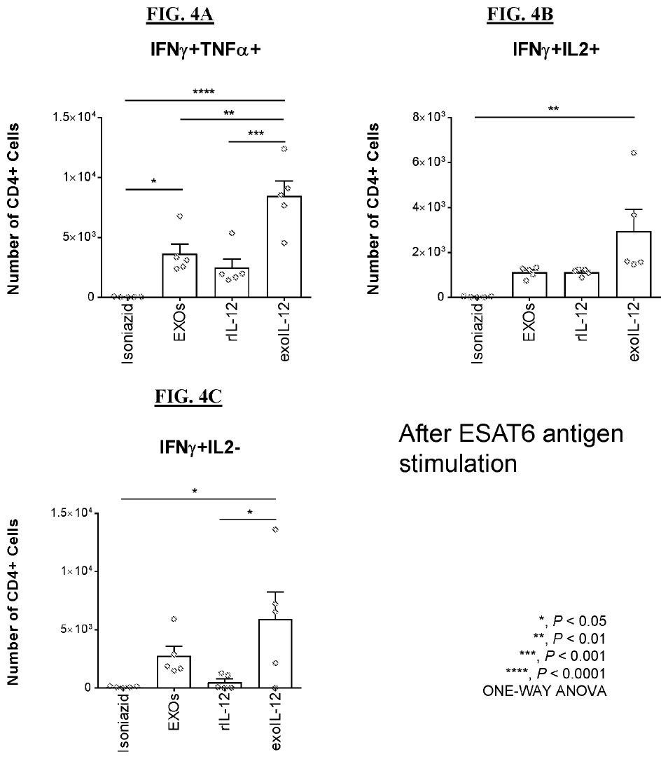

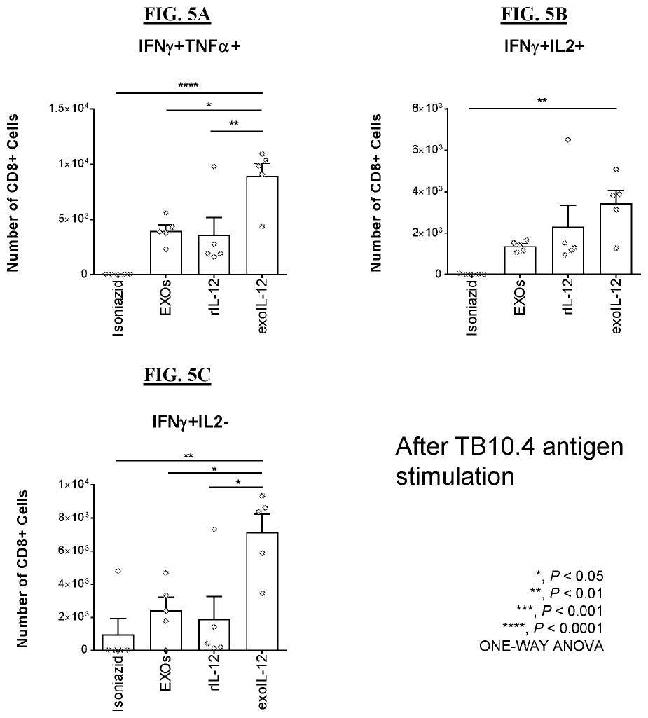

Administration of exoIL12 to a TB mouse model resulted in a significant induction of immune cells specific to the TB antigens ESAT6 (FIG. 3A) and TB10.4 (FIG. 3B), relative to mice administered the antibiotic isoniazid, an exosome lacking IL-12, and recombinant IL-12 (rIL-12). Further, a significant induction CD4 T-cells with effector function was observed in mice administered exoIL-12 following stimulation with ESAT6 antigen, as compared to control mice (FIGS. 4A-4C); and a significant induction CD8 T-cells with effector function was observed in mice administered exoIL-12 following stimulation with TB10.4 peptide, as compared to control mice (FIGS. 5A-5C).

### Example 2

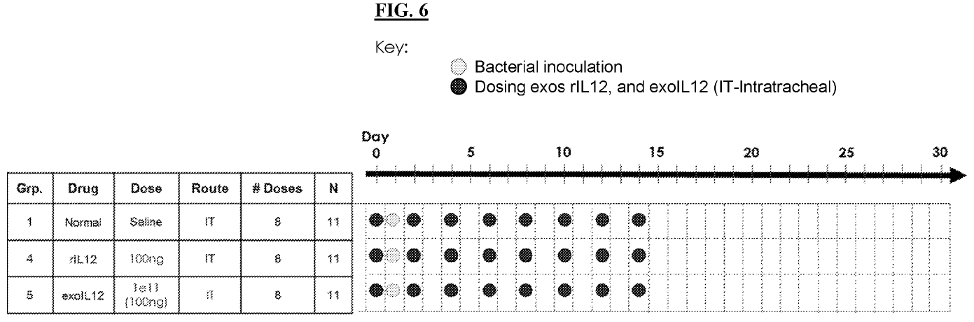

An in vivo study will be conducted to determine if exoIL12 can have an impact in a tuberculosis mouse model in prophylactic setting (FIG. 6). The endpoints of this study will be either measurement of viable bacterial burden and antigen specific effector T cells during Mycobacterium tuberculosis infection, or survival over time.

### Example 3

An in vivo study will be conducted to determine the efficacy of exoIL12 in a tuberculosis mouse model over an extended period of time. The endpoints of this study will be either measurement of viable bacterial burden and antigen specific effector T cells during Mycobacterium tuberculosis infection, or survival over time.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the invention(s). Many variations will become apparent to those skilled in the art upon review of this specification.

What is claimed:

1. A method of inducing an immune response against Mycobacterium tuberculosis in a subject in need thereof comprising administering an extracellular vesicle (EV) comprising Interleukin-12 (IL-12).

2. The method of claim 1, wherein the immune response is against one or more epitopes of Mycobacterium tuberculosis.

3. The method of claim 1 or 2, wherein the immune response is a cellular immune response, a humoral immune response, or both cellular and humoral immune responses.

4. The method of claim 3, wherein the induction of the cellular immune response, the humoral immune response, or both cellular and humor immune responses of the EV is increased by at least about 5%, at least about 10%, at least about 20%, at least about 30%, at least about 40%, at least about 50%, at least about 60%, at least about 70%, at least about 80%, at least about 90%, at least about 100% or more, compared to IL-12 without an EV.

5. The method of any one of claims 1 to 4, wherein the immune response is a CD4 T cell response, a CD8 T cell response, or both CD4 and CD8 T cell responses.

6. The method of any one of claims 2 to 5, wherein the epitope is an ESAT6 antigen, a TB10.4 antigen, or both ESAT6 antigen and TB10.4 antigen.

7. The method of claim 6, wherein the immune response is CD4 T-cell immune response with effector function that is specific to the ESAT6 antigen.

8. The method of claim 6 or 7, wherein the immune response is CD8 T-cell immune response that is specific to the TB10.4 antigen.

9. The method of any one of claims 6 to 8, wherein the ESAT6 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in
(SEQ ID NO: 370)
MTEQQWNFAGIEAAASAIQGNVTSIHSLLDEGKQSLTKLAAAWGGSGSE
AYQGVQQKWDATATELNN ALQNLARTISEAGQAMASTEGNVTGMFA.

10. The method of any one of claims 6 to 9, wherein the TB10.4 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in
(SEQ ID NO: 371)
MSQIMYNYPAMLGHAGDMAGYAGTLQSLGAEIAVEQAALQSAWQGDTGI
TYQAWQAQWNQAMEDLVRAYHAMSSTHEANTMAMMARDTAEAAKWGG.

11. The method of any one of claims 1 to 10, wherein the EV further comprises a TB antigen.

12. An extracellular vesicle (EV) comprising IL-12 and a TB antigen.

13. The EV of claim 12, wherein the EV induces an immune response against Mycobacterium tuberculosis in a subject in need thereof.

14. The EV of claim 13, wherein the immune response is against one or more epitopes of Mycobacterium tuberculosis.

15. The EV of claim 13 or 14, wherein the immune response is a cellular immune response, a humoral immune response, or both cellular and humoral immune responses.

16. The EV of any one of claims 13 to 15, wherein the induction of the cellular immune response, the humoral immune response, or both cellular and humor immune responses of the EV is increased by at least about 5%, at least about 10%, at least about 20%, at least about 30%, at least about 40%, at least about 50%, at least about 60%, at least about 70%, at least about 80%, at least about 90%, at least about 100% or more, compared to IL-12 without an EV.

17. The EV of any one of claims 13 to 16, wherein the immune response is a CD4 T cell response, a CD8 T cell response, or both CD4 and CD8 T cell responses.

18. The EV of any one of claims 14 to 17, wherein the epitope is an ESAT6 antigen, a TB10.4 antigen, or both ESAT6 antigen and TB10.4 antigen.

19. The EV of any one of claims 14 to 18, wherein the immune response is CD4 T-cell immune response with effector function that is specific to the ESAT6 antigen.

20. The EV of claim 18 or 19, wherein the immune response is CD8 T-cell immune response that is specific to the TB10.4 antigen.

21. The EV of any one of claims 18 to 20, wherein the ESAT6 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in
(SEQ ID NO: 370)
MTEQQWNFAGIEAAASAIQGNVTSIHSLLDEGKQSLTKLAAAWGGSGSE
AYQGVQQKWDATATELNNALQNLARTISEAGQAMASTEGNVTGMFA.

22. The EV of any one of claims 18 to 21, wherein the TB10.4 antigen comprises an epitope having at least three amino acids, at least four amino acids, at least five amino acids, at least six amino acids, at least seven amino acids, at least eight amino acids, at least nine amino acids, at least ten amino acids, at least eleven amino acids, at least twelve amino acids, at least thirteen amino acids, at least fourteen amino acids, at least fifteen amino acids of the amino acid sequence as set forth in
(SEQ ID NO: 371)
MSQIMYNYPAMLGHAGDMAGYAGTLQSLGAEIAVEQAALQSAWQGDTGI
TYQAWQAQWNQAMEDLVRAYHAMSSTHEANTMAMMARDTAEAAKWGG.

23. The method of claim 11 or the EV of any one of claims 18 to 22, wherein the TB antigen is identical to the ESAT6 antigen or the TB10.4 antigen.

24. The method of any one of claims 1 to 12 and 23 or the EV of any one of claims 12 to 23, wherein the EV further comprises a scaffold moiety.

25. The method or EV of claim 24, wherein the IL-12 is linked to the scaffold moiety.

26. The method or EV of claim 24, wherein the TB antigen is further linked to the scaffold moiety.

27. The method or EV of any one of claims 24 to 26, wherein the EV further comprises a second scaffold moiety.

28. The method or EV of claim 27, wherein the IL-12 is linked to the scaffold moiety, and the TB antigen is linked to the second scaffold moiety.

29. The method or EV of claim 27 or 28, wherein the first scaffold moiety and the second scaffold moiety are the same.

30. The method or EV of claim 27 or 28, wherein the first scaffold moiety and the second scaffold moiety are different.

31. The method or EV of any one of claims 25 to 30, wherein the first scaffold moiety is a Scaffold X.

32. The method or EV of any one of claims 25 to 30, wherein the first scaffold moiety is a Scaffold Y.

33. The method or EV of any one of claims 27 to 30, wherein the second scaffold moiety is a Scaffold Y.

34. The method or EV of any one of claims 27 to 30, wherein the second scaffold moiety is a Scaffold X.

35. The method or EV of claim 31 or 34, wherein Scaffold X is a scaffold protein that is capable of anchoring the IL-12 and/or the TB antigen on the luminal surface of the EV and/or on the exterior surface of the EV.

36. The method or EV of any one of claims 31, 34, and 35, wherein Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), and any combination thereof.

37. The method or EV of claim 36, wherein the first scaffold moiety is PTGFRN protein.

38. The method or EV of any one of claims 31, 34, and 35, wherein the first scaffold moiety comprises an amino acid sequence as set forth in SEQ ID NO: 33.

39. The method or EV of claim 37 or 38, wherein the first scaffold moiety comprises an amino acid sequence at least 50%, at least 60%, at least 70%, at least 80%, at least 85%, at least 90%, at least 95%, at least 96%, at least 97%, at least 98%, at least 99%, or about 100% identical to SEQ ID NO: 1.

40. The method or EV of claim 32 or 33, wherein Scaffold Y is a scaffold protein that is capable of anchoring the IL-12 and/or the TB antigen on the luminal surface of the EV.

41. The method or EV of claim 40, wherein the Scaffold Y is selected from the group consisting of myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), and any combination thereof.

42. The method or EV of any one of claims 32, 33, or 40, wherein the Scaffold Y is BASP1 protein.

43. The method or EV of any one of claims 32, 33, or 40 to 42, wherein the Scaffold Y comprises an N terminus domain (ND) and an effector domain (ED), wherein the ND and/or the ED are associated with the luminal surface of the EV.

44. The method or EV of claim 43, wherein the ND is associated with the luminal surface of the exosome via myristoylation.

45. The method or EV of claim 43 or 44, wherein the ED is associated with the luminal surface of the exosome by an ionic interaction.

46. The method or EV of any one of claims 43 to 45, wherein the ED comprises (i) a basic amino acid or (ii) two or more basic amino acids in sequence, wherein the basic amino acid is selected from the group consisting of Lys, Arg, His, and any combination thereof.

47. The method or EV of claim 45, wherein the basic amino acid is (Lys)n, wherein n is an integer between 1 and 10.

48. The method or EV of any one of claims 43 to 47, wherein the ED comprises Lys (K), KK, KKK, KKKK (SEQ ID NO: 205), KKKKK (SEQ ID NO: 206), Arg (R), RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof.

49. The method or EV of any one of claims 43 to 47, wherein the ND comprises the amino acid sequence as set forth in G:X2:X3:X4:X5:X6, wherein G represents Gly; wherein “:” represents a peptide bond, wherein each of the X2 to the X6 is independently an amino acid, and wherein the X6 comprises a basic amino acid.

50. The method or EV of claim 49, wherein:
(i) the X2 is selected from the group consisting of Pro, Gly, Ala, and Ser;
(ii) the X4 is selected from the group consisting of Pro, Gly, Ala, Ser, Val, Ile, Leu, Phe, Trp, Tyr, Gln and Met;
(iii) the X5 is selected from the group consisting of Pro, Gly, Ala, and Ser;
(iv) the X6 is selected from the group consisting of Lys, Arg, and His; or
(v) any combination of (i)-(iv).

51. The method or EV of any one of claims 43 to 47, wherein the ND comprises the amino acid sequence of G:X2:X3:X4:X5:X6, wherein
(i) G represents Gly;
(ii) “:” represents a peptide bond;
(iii) the X2 is an amino acid selected from the group consisting of Pro, Gly, Ala, and Ser;
(iv) the X3 is an amino acid;
(v) the X4 is an amino acid selected from the group consisting of Pro, Gly, Ala, Ser, Val, Ile, Leu, Phe, Trp, Tyr, Gln and Met;
(vi) the X5 is an amino acid selected from the group consisting of Pro, Gly, Ala, and Ser; and
(vii) the X6 is an amino acid selected from the group consisting of Lys, Arg, and His.

52. The method or EV of any one of claims 49 to 51, wherein the X3 is selected from the group consisting of Asn, Gln, Ser, Thr, Asp, Glu, Lys, His, and Arg.

53. The method or EV of any one of claims 43 to 52, wherein the ND and the ED are joined by a linker.

54. The method or EV of claim 53, wherein the linker comprises one or more amino acids.

55. The method of any one of claims 43 to 54, wherein the ND comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), (v) GGKLSKK (SEQ ID NO: 211), or (vi) any combination thereof.

56. The method or EV of claim 55, wherein the ND comprises an amino acid sequence selected from the group consisting of (i) GGKLSKKK (SEQ ID NO: 238), (ii) GGKLSKKS (SEQ ID NO: 239), (iii) GAKLSKKK (SEQ ID NO: 240), (iv) GAKLSKKS (SEQ ID NO: 241), (v) GGKQSKKK (SEQ ID NO: 242), (vi) GGKQSKKS (SEQ ID NO: 243), (vii) GGKLAKKK (SEQ ID NO: 244), (viii) GGKLAKKS (SEQ ID NO: 245), (ix) GGKLSKKK (SEQ ID NO: 238), (x) GGKLSKKS (SEQ ID NO: 239), and (xi) any combination thereof.

57. The method or EV of any one of claims 43 to 56, wherein the ND comprises the amino acid sequence GGKLSKK (SEQ ID NO: 211).

58. The method or EV of any one of claims 43 to 57, wherein the Scaffold Y is at least about 8, at least about 9, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 105, at least about 110, at least about 120, at least about 130, at least about 140, at least about 150, at least about 160, at least about 170, at least about 180, at least about 190, or at least about 200 amino acids in length.

59. The method or EV of claims 43 to 58, wherein the Scaffold Y comprises (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 291).

60. The method or EV of claims 43 to 58, wherein the Scaffold Y consists of (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 291).

61. The method or EV of claim 43 to 60, wherein the Scaffold Y does not comprise Met at the N terminus.

62. The method or EV of any one of claims 43 to 61, wherein the Scaffold Y comprises a myristoylated amino acid residue at the N terminus of the scaffold protein.

63. The method or EV of claim 62, wherein the amino acid residue at the N terminus of the Scaffold Y is Gly.

64. The method or EV of any one of claims 25 to 63, wherein the IL-12 is linked to a scaffold moiety (e.g., Scaffold X) on the exterior surface of the EV, and the TB antigen is linked to the scaffold moiety on the luminal surface of the EV.

65. The method or EV of any one of claims 25 to 63, wherein the IL-12 is linked to a scaffold moiety (e.g., Scaffold X) on the luminal surface of the EV, and the TB antigen is linked to the scaffold moiety on the exterior surface of the EV.

66. The method or EV of any one of claims 27 to 63, wherein the TB antigen is linked to a scaffold moiety (e.g., Scaffold X) on the exterior surface of the EV, and the IL-12 is linked to a second scaffold moiety (e.g., Scaffold X) on the exterior surface of the EV.

67. The method or EV of any one of claims 27 to 63, wherein the TB antigen is linked to a scaffold moiety (e.g., Scaffold X) on the exterior surface of the EV, and the IL-12 is linked to a second scaffold moiety (e.g., Scaffold Y) on the luminal surface of the EV.

68. The method or EV of any one of claims 27 to 63, wherein the IL-12 is linked to a scaffold moiety (e.g., Scaffold X) on the exterior surface of the EV, and the TB antigen is linked to a second scaffold moiety (e.g., Scaffold X) on the exterior surface of the EV.

69. The method or EV of any one of claims 27 to 63, wherein the IL-12 is linked to a scaffold moiety (e.g., Scaffold X) on the exterior surface of the EV, and the TB antigen is linked to a second scaffold moiety (e.g., Scaffold Y) on the luminal surface of the EV.

70. The method or EV of any one of claims 27 to 63, wherein the IL-12 is linked to a scaffold moiety (e.g., Scaffold Y) on the luminal surface of the EV, and the TB antigen is linked to a second scaffold moiety (e.g., Scaffold Y) on the luminal surface of the EV.

71. The method or EV of any one of claims 27 to 63, wherein the IL-12 is linked to a scaffold moiety (e.g., Scaffold X) on the luminal surface of the EV, and the TB antigen is linked to a second scaffold moiety (e.g., Scaffold Y) on the luminal surface of the EV.

72. The method or EV of any one of claims 25 to 71, wherein the IL-12 and/or the TB antigen is linked to the scaffold moiety by a linker.

73. The method or EV of any one of claims 25 to 31, wherein the IL-12 and/or the TB antigen is linked to the second scaffold moiety by a linker.

74. The method or EV of claim 72 or 73, wherein the linker is a polypeptide.

75. The method or EV of claim 72 or 73, wherein the linker is a non-polypeptide moiety.

76. The method of any one of claims 1 to 11 and 23 to 76 or EV of any one of claims 12 to 76, wherein the EV is an exosome.

77. A pharmaceutical composition comprising the EV of any one of claims 12 to 76 and a pharmaceutically acceptable carrier.

78. A cell that produces the EV of any one of claims 12 to 76.

79. A cell comprising one or more vectors, wherein the vectors comprises a nucleic acid sequence encoding the TB antigen in any one of claims 12 to 76, the IL-12 in any one of claims 12 to 76.

80. A kit comprising the EV of any one of claims 12 to 76 and instructions for use.

81. A method of making EVs comprising culturing the cell of claim 78 or 79 under a suitable condition and obtaining the EVs.

82. A method of inducing an immune response in a subject in need thereof comprising administering the EV of any one of claims 12 to 76 to the subject.

83. A method of preventing or treating a disease in a subject in need thereof, comprising administering the EV of any one of claims 12 to 76, wherein the disease is associated with the TB antigen.

84. The method of claim 83, wherein the disease is tuberculosis.

85. The method of any one of claims 1 to 11, 23 to 76, and 82 to 84, further comprising administering a TB antigen to the subject.

86. The method of claim 85, wherein the TB antigen is administered prior to, concurrently with, or after the administration of the EV.

87. The method of claim 85, wherein the subject is primed by the TB antigen.

88. The method of any one of claims 1 to 11, and 23 to 76, and 82 to 87, wherein the EV is administered parenterally, orally, intravenously, intramuscularly, intra-tumorally, intranasally, subcutaneously, or intraperitoneally.

89. The method of any one of claims 1 to 11, and 23 to 76, and 82 to 88, comprising administering an additional therapeutic agent.
